(19) United States
(12) Patent Application Publication
(10) Pub. No.: US 2023/0285970 A1
(43) Pub. Date: Sep. 14, 2023

(54) APPARATUSES WITH FLUID DROPLET GENERATORS COUPLED TO REACTION REGIONS AND FLUID EJECTORS

(71) Applicant: HEWLETT-PACKARD DEVELOPMENT COMPANY, L.P., Spring, TX (US)

(72) Inventors: Viktor SHKOLNIKOV, Palo Alto, CA (US); Alexander GOVYADINOV, Corvallis, OR (US)

(73) Assignee: HEWLETT-PACKARD DEVELOPMENT COMPANY, L.P., Spring, TX (US)

(21) Appl. No.: 17/692,589

(22) Filed: Mar. 11, 2022

(51) Int. Cl. B01L 3/00; C12Q 3/00; G01N 21/64

(52) U.S. Cl. CPC B01L 3/502761; C12Q 3/00; G01N 21/6428; B01L 2200/025; B01L 2200/027; B01L 2200/0652; B01L 2200/16; B01L 2300/0654; B01L 2400/02; G01N 2021/6439

(57) ABSTRACT

An example apparatus comprises a first microfluidic channel fluidically coupled to a first reservoir containing a carrier fluid, the first microfluidic channel including a reaction region, a fluid droplet generator, and a fluid ejector fluidically coupled to the first microfluidic channel and disposed downstream from the reaction region of the first microfluidic channel. The fluid droplet generator includes a portion of the first microfluidic channel and a second microfluidic channel that intersects the first microfluidic channel and is fluidically coupled to a second reservoir containing a reaction fluid, where the reaction fluid including a plurality of cells and fluorescently-labeled capture reagents to form reaction products with a target molecule secreted by the plurality of cells.

### BACKGROUND

Targets within samples may be biochemically reacted to form a reaction product using different types of apparatuses and devices. Example biochemical reactions include nucleic acid amplification, antibody and antigen binding, ligation, among other types of reactions. The resulting reaction product may be detected to identify a presence of the target within the sample, to perform further reactions or operations, to develop biologic therapeutics, and for other purposes.

### BRIEF DESCRIPTION OF THE DRAWINGS

FIGS. 1A-1E illustrate an example apparatus including a fluid droplet generator coupled to a reaction region and a fluid ejector, in accordance with the present disclosure.

FIGS. 2A-2C illustrate other example apparatuses including a microfluidic device, a controller, and an optics system, in accordance with the present disclosure.

FIGS. 3A-3E illustrate different example apparatuses including a fluid droplet generator coupled to a reaction region and a fluid ejector, in accordance with the present disclosure.

FIG. 4 illustrates an example microfluidic device including a portion of an optics system in a reaction region, in accordance with the present disclosure.

FIGS. 5A-5D illustrate different example microfluidic devices and a portion of an optics system in a reaction region, in accordance with the present disclosure.

FIG. 6 illustrates an example method for detecting a reaction product, in accordance with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Biochemical reactions may occur between components of a sample and reagents. In many instances, living cells from a sample may secrete different molecules, such as antibodies, proteins, and metabolites. Devices and assessments or tests may be designed to implement a particular biochemical reaction with a target molecule secreted by cell, such as a specific antibody secreted by cells. By designing the device to implement the particular biochemical reaction, the target molecule may be detected as being present and secreted by the cell and/or the cell may be used for further processing and/or analysis. For example, the cell may be used for the development of biologic therapeutics, such as monoclonal antibodies.

For some applications, volumes of cells may be analyzed to identify respective cells which secrete the target molecule. Assessing volumes of cells may be difficult. As an example, for monoclonal antibodies, hybridoma cells may be first produced by using a number of b-cells that produce different antibodies and fusing the b-cells with myeloma cells. The hybridoma cells are then screened to identify cells that secrete a target antibody specific to an antigen. In many instances, not limited to monoclonal antibody production, selecting cells which secrete a target molecule may involve several cycles of dilution of the cells into different subsets, which are grown out, and assayed to test for binding until respective cells producing the target molecule are identified. Examples of the present disclosure are directed to apparatuses, microfluidic devices, and methods for detecting and sorting cells that secrete a target molecule while reducing the time for assessment as the cells may not be diluted and regrown, by obtaining fluorescence signals from fluorophores in response to a reaction between the target molecule and a fluorescently-labeled capture reagent. The process may be automated, with high-throughput, and which allows for convergence to a subset of cells with target properties, without growing the cells out. In some examples, the cells may be sorted using an integrated microfluidic device, which may allow for identifying different targets in parallel.

An example apparatus in accordance with the present disclosure comprises a first microfluidic channel fluidically coupled to a first reservoir containing a carrier fluid, a fluid droplet generator, and a fluid ejector fluidically coupled to the first microfluidic channel and disposed downstream from the reaction region of the first microfluidic channel. The first microfluidic channel including a reaction region. The fluid droplet generator including a portion of the first microfluidic channel, and a second microfluidic channel that intersects the first microfluidic channel and is fluidically coupled to a second reservoir containing a reaction fluid. The reaction fluid including a plurality of cells and fluorescently-labeled capture reagents to form reaction products with a target molecule secreted by the plurality of cells.

In some examples, the target molecule is a protein selected from the group consisting of: an antibody, an enzyme, a cytokine, a hormone, a metabolic product, a metabolite, a synthetic precursor, and a toxin. And, the fluorescently-labeled capture reagents is a molecule selected from the group consisting of: an antibody, an aptamer, and an antigen molecule specific to the target molecule.

In some examples, the fluid ejector includes a nozzle and a fluidic actuator fluidically coupled to the nozzle, the fluidic actuator to actuate to cause flow of fluid.

In some examples, the first microfluidic channel, the second microfluidic channel, and the fluid ejector are integrated on a microfluidic device, and the apparatus further includes a fluid dispensing device to house the microfluidic device, and including a controller communicatively coupled to the fluid ejector to selectively actuate the fluidic actuator of the fluid ejector to cause flow of the carrier fluid coordinated with flow of the reaction fluid to generate fluid droplets of the reaction fluid.

In some examples, the apparatus further includes a substrate, wherein the fluid ejector is to selectively eject the fluid droplets of the reaction fluid from the microfluidic device to a plurality of regions of the substrate, and a stage coupled to the substrate, wherein the controller is communicatively coupled to the stage to instruct the stage to move the substrate relative to the fluid ejector, such that the fluid ejector is aligned with a select region of the plurality of regions of the substrate.

In some examples, the apparatus further includes an optics system to provide polarized excitation light toward the reaction region. For example, wherein the first microfluidic channel, the second microfluidic channel, the fluid ejector, and a portion of the optics system are integrated on a microfluidic device, the portion including a bandpass filter disposed on a surface of reaction region to pass fluorescence light emitted from the reaction region within a wavelength range, a set of polarizers disposed on the bandpass filter and exposed to the first microfluidic channel within the reaction region, and circuitry coupled to the bandpass filter.

In some examples, the optics system is coupled to the reaction region and includes a light source to provide the excitation light toward the reaction region, a set of polarizers to polarize the excitation light from the light source to a first polarization, a bandpass filter to pass fluorescence light emitted from the reaction region within a wavelength range, and circuitry to measure fluorescence anisotropy based on the polarization of the fluorescence light emitted relative to the excitation light.

In some examples, the apparatus further includes a waste chamber fluidically coupled to the second microfluidic channel.

In various examples of the present disclosure, a microfluidic device comprises a first microfluidic channel fluidically coupled to a first reservoir containing a carrier fluid, a second microfluidic channel that intersects the first microfluidic channel and is fluidically coupled to a second reservoir containing a reaction fluid, a bandpass filter disposed within the reaction region, a set of polarizers disposed on the bandpass filter and exposed to the first microfluidic channel within the reaction region, and a fluid ejector fluidically coupled to and disposed within the first microfluidic channel and downstream from the reaction region to eject fluid droplets of the reaction fluid from the first microfluidic channel. The first microfluidic channel including a reaction region. And, the reaction fluid including a plurality of cells and fluorescently-labeled capture reagents to form reaction products with a target molecule secreted by the plurality of cells, wherein a fluid droplet generator is formed at the intersection of the first microfluidic channel and the second microfluidic channel.

In some examples, the first microfluidic channel is to pass an excitation light through and toward the reaction region from a light source, and wherein the set of polarizers are to selectively select polarization of fluorescence light emitted from the reaction region as illuminated by the excitation light to a first polarization and to a second polarization, and the bandpass filter is to block the excitation light and pass the fluorescence light emitted from the reaction region.

In some examples, the microfluidic device further includes circuitry coupled to the bandpass filter to provide a fluorescence anisotropy measurement based on the polarization of the fluorescence light emitted relative to the excitation light, and a controller communicatively coupled to the circuitry and the fluid ejector to cause flow of fluid, including the fluid droplets of the reaction fluid as carried by the carrier fluid, toward the reaction region of the first microfluidic channel, and selectively eject the fluid droplets of the reaction fluid based on the fluorescence anisotropy measurement.

In some examples, the circuitry includes a set of diodes coupled to the bandpass filter and signal processing circuitry coupled to the set of diodes.

In various examples of the present disclosure, a method comprises flowing a carrier fluid from a first reservoir to and along a portion of a first microfluidic channel of a microfluidic device, and flowing a reaction fluid from a second reservoir to a second microfluidic channel of the microfluidic device and into the first microfluidic channel that intersects the second microfluidic channel, the reaction fluid including a plurality of cells and fluorescently-labeled capture reagents to form reaction products with a target molecule secreted by the plurality of cells. The method further comprises forming fluid droplets of the reaction fluid via an intersection of the flow of the carrier fluid and the flow of the reaction fluid, flowing the fluid droplets of the reaction fluid to a reaction region of the first microfluidic channel, providing polarized excitation light toward the reaction region using an optics system, detecting reaction products from a biochemical reaction between the target molecule and the fluorescently-labeled capture reagents by measuring fluorescence anisotropy based on a polarization of florescence light emitted from the reaction region as illuminated by the polarized excitation light, and selectively ejecting the fluid droplets of the reaction fluid, that are associated with the detected reaction products, from the microfluidic device to a substrate via a fluid ejector of the microfluidic device.

In some examples, the method further includes selectively flowing the remaining fluid droplets of the reaction fluid to one of a waste region and a recycling region.

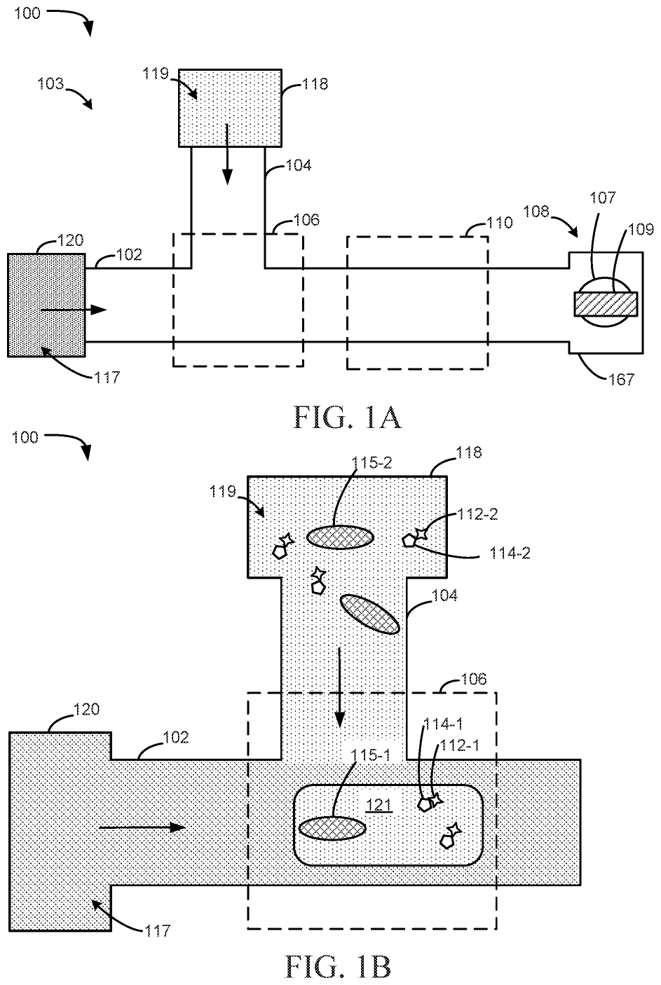

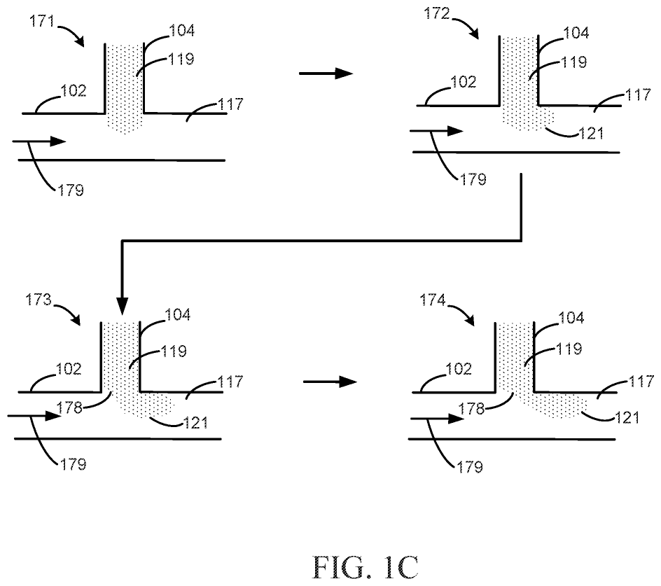

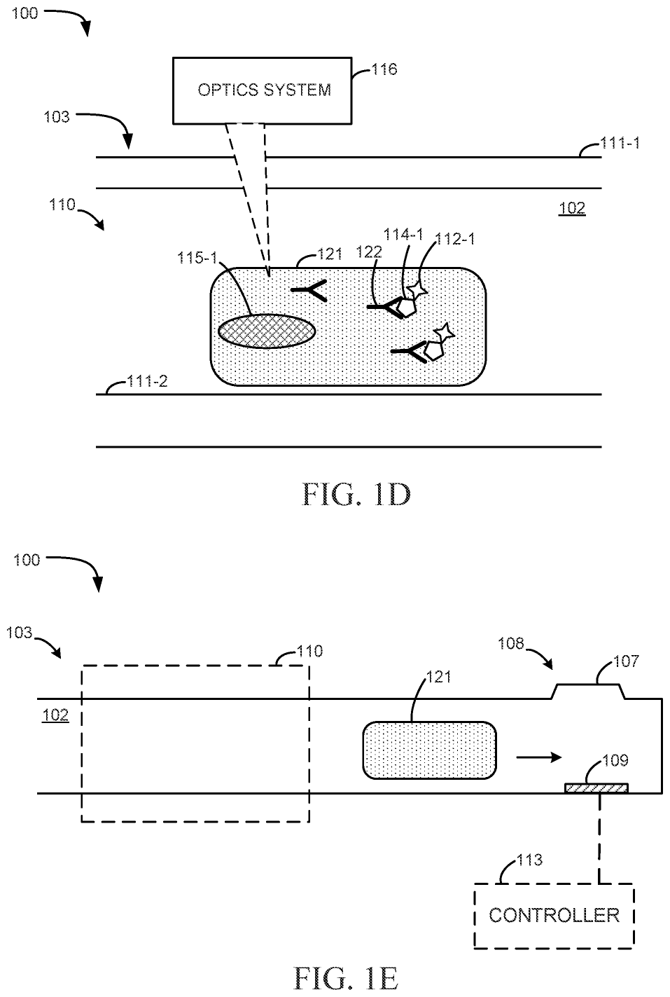

Turning now to the figures, FIGS. 1A-1E illustrate an example apparatus including a fluid droplet generator coupled to a reaction region and a fluid ejector, in accordance with the present disclosure. The apparatus 100 illustrated by FIGS. 1A-1E may be used to flow a reaction fluid 119 therethrough, with the reaction fluid 119 containing cells which may secrete molecules of interest, sometimes herein referred to as a “target molecule”, which is a molecule secreted by a cell. The target molecule may be an antibody, an enzyme, a hormone, a metabolite, a metabolic product, a synthetic precursor, a toxin, among other molecules secreted by the cell and targeted for detection.

Referring to FIG. 1A, an example apparatus 100 comprises a first microfluidic channel 102 which may be coupled to a first reservoir 120 containing a carrier fluid 117. The apparatus 100 may further comprise a second microfluidic channel 104 that intersects the first microfluidic channel 102 and is fluidically coupled to a second reservoir 118 containing a reaction fluid 119. As used herein, a microfluidic channel includes and/or refers to a path through the apparatus 100 which a fluid or semi-fluid may pass, which may allow for transport of volumes of fluid on the order of microliters (82 L), nanoliters, picoliters, or femtoliters, and which may be formed of an etched or micromachined portion (e.g., negative space formed in a substrate).

The reaction fluid 119 includes fluid containing cells and fluorescently-labeled captured reagents. In some examples, the reaction fluid 119 may include a sample mixed or dispersed in an aqueous solution. A sample, as used herein, includes and/or refers to any biological material collected, such as from a subject or other source. In some examples, the reaction fluid 119 may be formed of a first reaction fluid containing the plurality of cells and a second reaction fluid containing the fluorescently-labeled captured reagents. The first and second reaction fluids may be mixed in the second reservoir 118 or in the second microfluidic channel 104. A carrier fluid 117, as used herein, includes and/or refers a fluid that flows through portions of the apparatus 100 and which carries solid and/or fluid particles, such as fluid droplets of the reaction fluid 119. Fluorescently-labeled capture reagents, as used herein, include and/or refer to reagents, such as substances, molecules, or other components, that bind or are complementary to the target molecule secreted by cells in the reaction fluid 119 and bound to a fluorophore.

As previously described, the reaction fluid 119 may include a plurality of cells and fluorescently-labeled capture reagents which may form reaction products with a target molecule secreted by the plurality of cells. A reaction product, as used herein, includes and/or refers to a species or substance formed from a biochemical reaction between the target molecule and the fluorescently-labeled capture reagents.

In some examples, the reaction fluid 119 may include an aqueous fluid and the carrier fluid 117 may include an oil fluid. For example, the carrier fluid 117 may include an oil. In some examples, the carrier fluid 117 may include a silicon oil or fluorinated oil, such as FC-40 or FC-3283. Non-limiting examples of the carrier fluid 117 include FC-40, FC-43, FC-77, fluorophoroheptane (FC-84), FC-3283, perfluoro-n-octane, perfluorodecalin, perfluorophenanthrene, perfluorohexyloctane, octofluoropropane, decafluorobutane, perfluoropentane, perfluorohexane, perfluorooctane, decafluoropentane, perfluoro(2-methyl-3-pentaone), perfluoro-15-crown-5-ether, bis-(perfluorobutyl) ethane, perfluorobutyl tetrahydrofuran, bi-perfluorohexyl ethane, perfluoro-n-hexane, perfluorooctyl bromide, perfluorotributylamine, perfluorotripentylamine, and perfluorotripropylamine, among others. In some examples, the carrier fluid 117 may include a non-fluorinated oil, such as polyphenylmehtylsiloxane, polydimethylsiloxane, hexadecane, tetradecane, octadecane, dodecane, mineral oil, isopar, or squalene. However examples are not so limited and may include other types of carrier fluids and reaction fluids that are immiscible.

The apparatus 100 further includes a fluid droplet generator 106. A fluid droplet generator includes and/or refers to circuitry and/or a physical structure used to form fluid droplets of the reaction fluid 119. As shown by FIG. 1A, the fluid droplet generator 106 includes a portion of the first microfluidic channel 102 and the second microfluidic channel 104 (or a portion thereof). A fluid droplet of the reaction fluid 119, as used herein, includes and/or refers to a discrete portion of reaction fluid 119 (e.g., a liquid), which may be surrounded by the carrier fluid 117. As an example of a fluid droplet of the reaction fluid 119, an immiscible fluid, such as an aqueous solution, is surrounded by an oil phase. Further description and illustration of fluid droplet formation is provided below in connection with FIGS. 1B-1C.

The first microfluidic channel 102 includes a reaction region 110. Referring to FIG. 1D, the reaction region 110 may be used to perform a biochemical reaction associated with a target molecule 122 in a fluid droplet 121 of the reaction fluid and fluorescently-labeled capture reagents 112-1, 114-1. As used herein, a reaction region includes and/or refers to a portion of the first microfluidic channel which is downstream from the fluid droplet generator and upstream from a fluid ejector, in which a biochemical reaction may occur and may be visualized using an optics system, such as the optics system 116 illustrated by FIG. 1D. The reaction region 110 may have a surface that includes with a transparent window to allow light to pass through, as further described herein.

Referring back to FIG. 1A, the apparatus 100 further includes a fluid ejector 108. The fluid ejector 108 is coupled to the first microfluidic channel 102 and disposed downstream from the reaction region 110 of the first microfluidic channel 102. As used herein, a fluid ejector includes and/or refers to a physical structure, such as a firing chamber 167, to receive a fluid, such as from a manifold, fluid slot, or fluid hole array. The fluid ejector 108 may include a nozzle 107 and a fluidic actuator 109 fluidically coupled to the nozzle 107. The fluidic actuator 109 may be disposed in the firing chamber 167 coupled to the nozzle 107 and the first microfluidic channel 102, and is positioned in line with the nozzle 107. For instance, the fluidic actuator 109 may be positioned directly above or below the nozzle 107. A fluidic actuator, as used herein, includes and/or refers to circuitry and/or a physical structure that causes movement of fluid. Actuation of the fluidic actuator 109 may cause some fluid contained in the first microfluidic channel 102 to be dispensed or expelled out of the nozzle 107. The fluidic actuator 109 may be actuated via application of a voltage or current, as further described below. A firing chamber includes and/or refers to a semi-enclosed region of the apparatus 100 (e.g., a microfluidic device) fluidically coupled to the first microfluidic channel 102, with the nozzle 107 and fluidic actuator 109 disposed within and/or through a surface of the firing chamber 167.

Example fluidic actuators include electrodes, a fluidic pump, a magnetostrictive element, an ultrasound source, mechanical/impact driven membrane actuators, and magneto-restrictive drive actuators, among others. Example fluidic pumps include a piezo-electric pump and a resistor, such as a thermal inkjet resistor (TIJ).

In some examples, the fluidic actuator 109 includes a piezoelectric-based pump. The piezoelectric-based pump may generate pressure pulses that force fluid droplets of the reaction fluid 119 out of the nozzle 107. In such piezoelectric-based pumps, a voltage may be applied to the fluidic actuator 109 that is in the form of a piezoelectric element (e.g., piezoelectric material) located in the fluid ejector 108. When a voltage is applied, the piezoelectric element changes shape, which generates a pressure pulse that forces a fluid droplet of the reaction fluid 119 from the fluid ejector 108.

In some examples, the fluidic actuator 109 includes a TIJ resistor. Activation of the TIJ resistor may create the flow of fluid by firing fluid droplets of the reaction fluid 119 from the first microfluidic channel 102 and/or creating a vapor bubble. The TIJ resistor may create bubbles that force the fluid droplets of the reaction fluid 119 out of the first microfluidic channel 102. For example, a pulse of current may be passed through the fluid ejector 108 in the form of a TIJ resistor positioned in the fluid ejector 108. The TIJ resister acts as a heater, and heat from the TIJ resistor causes a vaporization of the reaction fluid 119 in the fluid ejector 108 to form the vapor bubble, which causes a pressure increase that propels the fluid droplet of the reaction fluid 119.

Examples are not so limited and additional and/or different types of fluid ejectors may be used to eject fluid from the first microfluidic channel 102. Similarly, different and/or additional components may be coupled to apparatus 100 for processing biochemical reactions.

In various examples, the fluidic actuator 109 may be actuated to cause flow of fluid within the apparatus 100. For example, the flow of fluid may include selectively ejecting fluid droplets of the reaction fluid 119 and/or the carrier fluid 117. As further described below, in some examples, the actuation of the fluidic actuator 109 may be used to form the fluid droplets of the reaction fluid 119 as carried by the carrier fluid 117.

In some examples, the first microfluidic channel 102, the second microfluidic channel 104, and the fluid ejector 108 are integrated on a microfluidic device 103. As illustrated by and referring to FIG. 1D, the microfluidic device 103 may include a housing formed by a first substrate 111-1 and a second substrate 111-2, with the first microfluidic channel 102 including the reaction region 110 and the second microfluidic channel (not illustrated by FIG. 1D, among other components) formed by and/or between the substrates 111-1, 111-2 as etched or micromachined portions. Each substrate 111-1, 111-2 may be formed of a plurality of different materials which are in layers, e.g., layers of substrates, in stack, as further described herein. Accordingly, the microfluidic channels 102, 104, chambers, and other components may be defined by surfaces fabricated in the substrate(s) 111-1, 111-2 of the microfluidic device 103.

In some examples, at least one of the substrate 111-1, 111-2 of the microfluidic device 103 may include or form a lid. The lid may be comprised of any suitable material, and a non-limiting example material includes SU-8. In some examples, the lid or a portion of the lid may be formed of a transparent material, such that excitation light and emitted light may pass through. In some examples, the lid may have a transparent window area which may allow light to pass through.

Referring back to FIG. 1A, in some examples, the first reservoir 120 and/or second reservoir 118 may be integrated on the microfluidic device 103. For example, the first reservoir 120 and/or second reservoir 118 may respectively fluidically couple to the first microfluidic channel 102 and the second microfluidic channel 104 through manifolds. In other examples, the first reservoir 120 and/or second reservoir 118 may be separate from the microfluidic device 103, such as being integrated on a second microfluidic device which is used to insert the fluids 117, 119 from the second microfluidic device (e.g., a cartridge) to the microfluidic device 103, as further described herein. As used herein, a reservoir includes and/or refers to container coupled to the microfluidic device 103 or an enclosed and/or a semi-enclosed region of the microfluidic device 103 that stores a fluid for chemical processing by the microfluidic device 103.

FIGS. 1B-1E illustrate example operation of the apparatus 100 of FIG. 1A. The common features of the apparatus 100, which are similarly labeled, are not repeated for ease of reference.

FIG. 1B shows a close-up view of the fluid droplet generator 106 of the apparatus 100 and corresponding fluid droplet formations. As shown by FIG. 1B, the first reservoir 120 contains the carrier fluid 117 and is fluidically coupled to the first microfluidic channel 102. The second reservoir 118 contains the reaction fluid 119 and is fluidically coupled to the second microfluidic channel 104. The reaction fluid 119 includes a plurality of cells 115-1, 115-2 and a plurality of fluorescently-labeled capture reagents that include reagents 114-1, 114-2 bound to fluorophores 112-1, 112-2.

Referring back to FIG. 1A, in some examples, the fluid ejector 108 may cause flow of the carrier fluid 117 and the reaction fluid 119. For example, the fluidic actuator 109 of the fluid ejection 108 may be actuated to cause pulling forces on both fluids 117, 119, and in response, cause a first flow of the carrier fluid 117 (as illustrated by the arrow) and a second flow of the reaction fluid 119 (as illustrated by the arrow). The first flow of the carrier fluid 117 and the second flow of the reaction fluid 119 may intersect and cause the formation of fluid droplets of the reaction fluid 119, as illustrated by fluid droplet 121 of FIG. 1B. For example, the second flow of the reaction fluid 119 may form a cross-flow or an angle-flow with respect to the first flow of the carrier fluid 117, which causes formation of fluid droplets of the reaction fluid 119. In such examples, fluid is drawn from the two microfluidic channels 102, 104, and the reaction fluid 119 is immiscible with the carrier fluid 117, which allows for the reaction fluid 119 to segregate into fluid droplets of the reaction fluid 119 with the carrier fluid 117 spacing therebetween and around the fluid droplets of the reaction fluid 119.

FIG. 1C shows an example operation of the fluid droplet generator 106, as illustrated by FIG. 1B, forming a fluid droplet 121 from the reaction fluid 119. As shown at 171, the carrier fluid 117 is flowing as a first flow in the first microfluidic channel 102, as illustrated by arrow 179, as the reaction fluid 119 enters the first microfluidic channel 102 from the second microfluidic channel 104. As previously described, the reaction fluid 119 flows as a second flow that intersects the first flow of the carrier fluid 117. As shown at 172, the reaction fluid 119 expands into the first microfluidic channel 102 and starts forming a fluid droplet 121 of the reaction fluid 119. As shown at 173, as the reaction fluid 119 continues to expand into the first microfluidic channel 102, a neck shape 178 forms, and as shown at 174, the fluid droplet 121 of the reaction fluid 119 separates from the remaining portion of the reaction fluid 119 by breaking off at the neck shape 178.

The hydraulic diameter of the first microfluidic channel 102 and the second microfluidic channel 104, along with lengths of the microfluidic channels 102, 104, may set the flow rate within the microfluidic channels 102, 104 and thereby set the size of the fluid droplet 121 of the reaction fluid 119 and spacing between respective fluid droplets of the reaction fluid 119. For example, the velocity of the carrier fluid 117 may be defined as:

$${u}_{d / 2} \approx 2 {u}_{c} ( 1 - {( \frac{{d}_{c} - d}{{d}_{c}} )}^{2} ) ,$$

where d_{c} is the diameter of the first microfluidic channel 102 and d is the diameter of the second microfluidic channel 104. For example, for rectangular channel geometries, d_{c}=2w_{c}h/(w_{c}+h), where w_{c} is the width of the first microfluidic channel 102 and h is the height of the first microfluidic channel 102. Further, u refers to the velocity vector, with u_{c} referring to the velocity vector of the carrier fluid 117 and u_{d} referring to the velocity of the reaction fluid 119, as respectively input to the fluid droplet generator 106. The fluid droplet generator 106 may be operating in the low Reynolds number regime, and the fluid droplet 121 of the reaction fluid 119 may experience drag force. If the fluid droplet 121 of the reaction fluid 119 is a sphere, the drag force may be defined as:

F_{D}≈3πμ_{c}d(u_{d/2}−u),

where u is the fluid droplet 121 velocity and u_{d/2}−u accounts for the reduction of the drag force due to motion of the fluid droplet 121 of the reaction fluid 119 relative to the carrier fluid 117. This relation may be an approximate relation, as the relation may not account for the fluid droplet 121 of the reaction fluid 119 interacting with surfaces of the microfluidic channels 102, 104 and assumes the fluid droplet 121 of the reaction fluid 119 is solid. Fora fluid droplet 121 of the reaction fluid 119 that is liquid with a viscosity that is different than the carrier fluid 117, the drag force FD may be defined as:

$${F}_{D} \approx 3 {πμ}_{C} d ( {u}_{d / 2} - u ) \frac{1 + 2 α / 3}{1 + α} ,$$

where a=μ_{c}/μ_{d}, and where μ_{c} is the dynamic viscosity of the carrier fluid 117 and μ_{d} is the dynamic viscosity of the reaction fluid 119. The interfacial tension force F_{γ}on the breaking off the fluid droplet 121 of the reaction fluid 119 may be estimated to be on an order of:

F_{γ}˜πγw_{d,}

where w_{d} is the width of the second microfluidic channel 104. The carrier fluid velocity may be substituted into the drag force F_{D} calculation to provide a scaling for the resulting diameter of the fluid droplet 121 of the reaction fluid 119 as:

$$3 {πμ}_{C} d ( 2 {u}_{c} ( 1 - {( \frac{{d}_{c} - d}{{d}_{c}} )}^{2} ) - u ) β - {πγ w}_{d} = 0 ,$$

where:

$$β = \frac{1 + 2 α / 3}{1 + α} .$$

The calculation of the size of the fluid droplet 121 of the reaction fluid 119 may be further nondimensionalized.

Referring back to FIG. 1B, each fluid droplet of the reaction fluid 119, as shown by the fluid droplet 121, may include a cell 115-1 and a subset of the plurality of fluorescently labeled capture reagents 112-1, 114-1. The fluorescently-labeled capture reagent 112-1, 112-2, 114-1, 114-2 are herein sometime referred to as “capture reagents 112, 114” for ease of reference. The capture reagents 112, 114 are detectable due to the fluorophores 112-1, 112-2 bound thereto and which emit a detectable fluorescent signal. In some examples, each fluid droplet of the reaction fluid 119 may contain a single cell, however examples are not so limited.

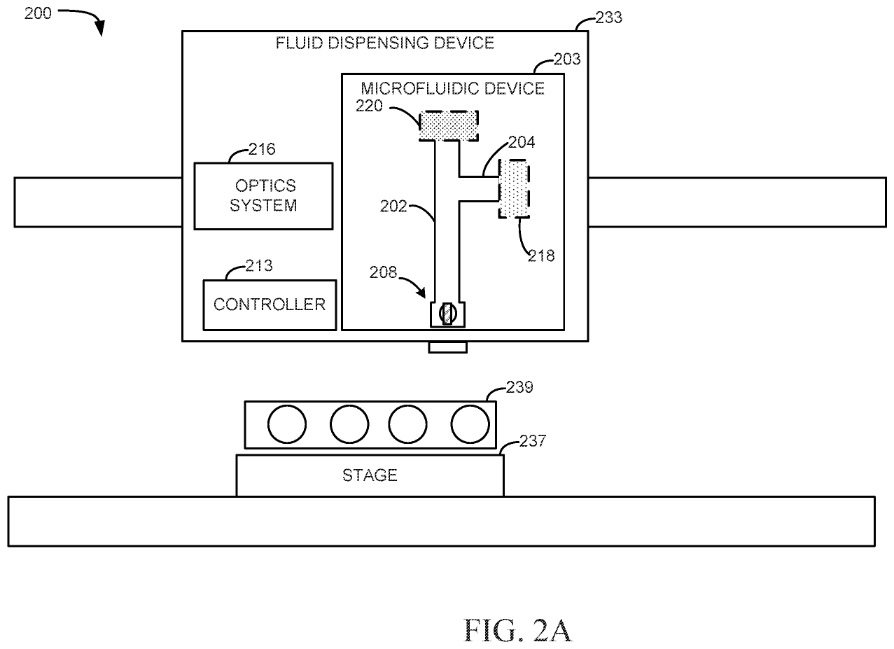

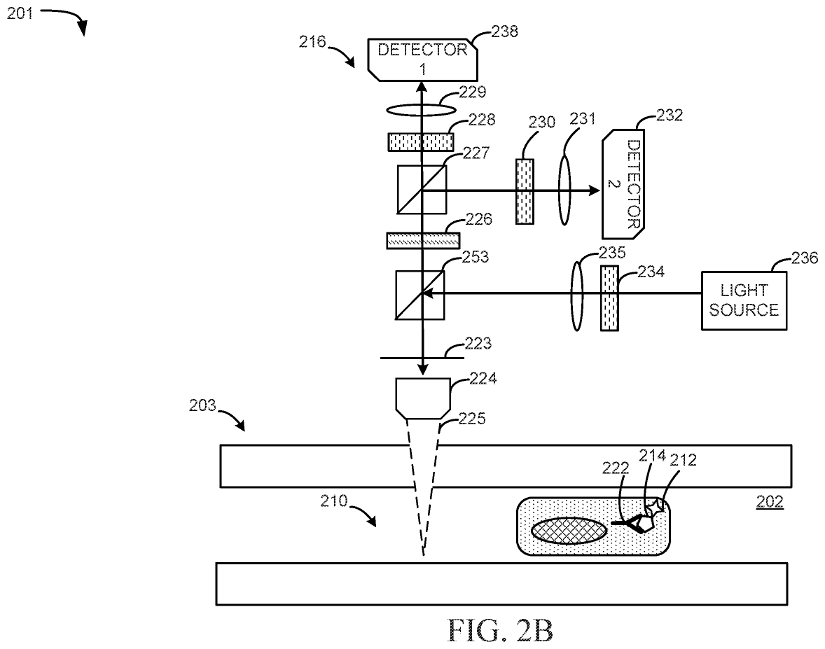

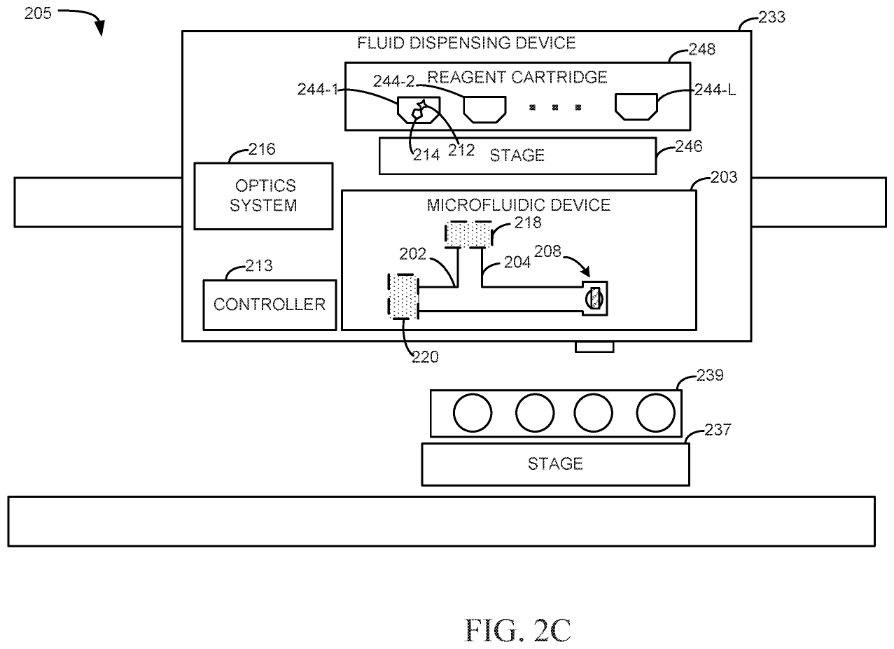

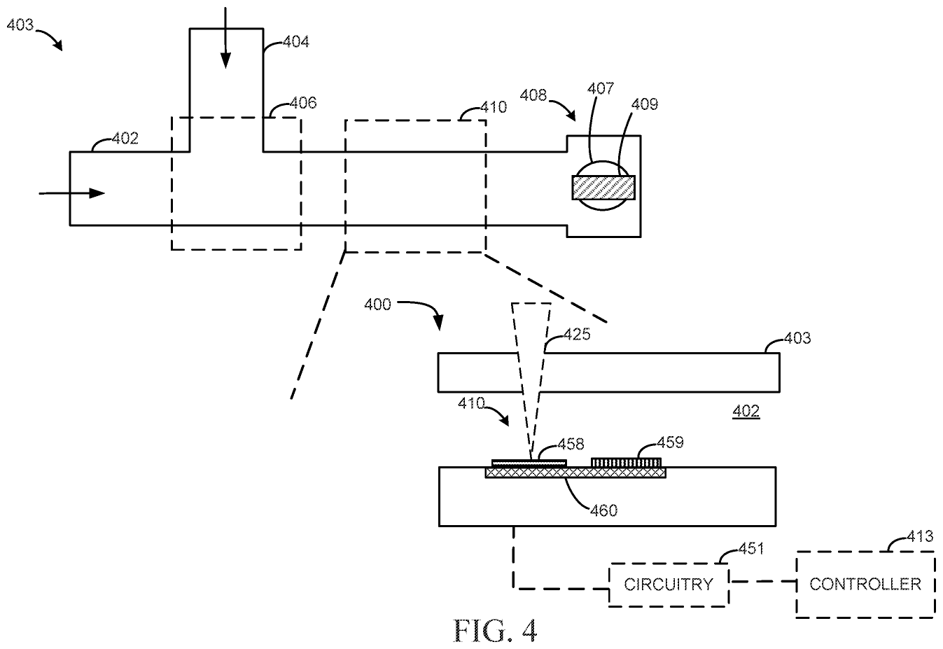

FIG. 1D shows a close-up view of the reaction region 110 of the apparatus 100 of FIG. 1A. In some examples, the apparatus 100 may further include an optics system 116. The optics system 116 may provide polarized excitation light toward the reaction region 110. The optics system 116 may further measure polarization of fluorescent light emitted from the reaction region 110 as illuminated by the polarized excitation light. Components of the optics system 116 are further illustrated herein and described with reference to at least FIG. 2B and FIG. 4.

As shown by the example of FIG. 1D, a fluid droplet 121 of the reaction fluid is flown into the reaction region 110. The fluid droplet 121 of the reaction fluid includes a cell 115-1 and the respective fluorescently-labeled capture reagent 112-1, 114-1. The cell 115-1 may be incubated with the fluorescently-labeled capture reagents 112-1, 114-1, which results in the cell 115-1 secreting molecules. The cell 115-1 may naturally secrete molecules while in the reaction region 110, in some example. In some examples, the secretion may be controlled and/or activated by light or by exposure to a signaling molecule.

In some examples, the cell 115-1 secretes a target molecule 122 that the fluorescently-labeled capture reagent 112, 114 has an affinity for. In response, the target molecule 122 binds to the respective fluorescently-labeled capture reagent 112, 114 to form a reaction product. The target molecule 122 may have a mass that is at least equal to the fluorescently-labeled capture reagent 112, 114. In response to binding, a reaction product is formed that has a greater mass than the fluorescently-labeled capture reagents 112, 114.

Example target molecules include an antibody, an enzyme, a cytokine, a hormone, a metabolic product, a metabolite, a synthetic precursor, and a toxin. Example fluorescently-labeled capture reagents include an antibody, an aptamer, and an antigen molecule specific to the target molecule. As a specific example, the target molecule 122 includes an antibody that binds to a target antigen and the fluorescently-labeled capture reagents 112, 114 include molecules exhibiting the target antigen on a surface. Such examples may be used to identify cells that secrete antibodies specific to a target, such as targets associated with a virus, bacteria, cancer or other disease. As another example, the target molecule 122 includes a metabolic product secreted by the cell 115-1 and the fluorescently-labeled capture reagents 112, 114 include antibodies that bind to the metabolic product.

The optics system 116 may be used to observe the reaction region 110 while binding occurs. The optics system 116 may provide polarized excitation light toward the reaction region 110 and measure a polarization of florescence light emitted from the reaction region 110 as illuminated by the polarized excitation light. The measured polarization of the florescence light emitted may be used to detect a reaction product, such as a signal from the fluorophore 112-1 indicating the capture reagent 114-1 is bound to the target molecule 122. As further described below in connection with FIG. 2B, the reaction product may be heavier than the fluorescently-labeled capture reagents 112-1, 114-1, which causes a change in a fluorescent anisotropy measure obtained using the optics system 116 and is used to detect the presence of the reaction product.

In some examples, the optics system 116 is coupled to the reaction region 110, such as being coupled to the microfluidic device 103. For example and referring to FIG. 2B, the optics system 216 includes a light source 236 to provide the excitation light toward the reaction region 210, a set of polarizers 228, 230, 234 to polarize the excitation light from the light source 236 to a first polarization, a bandpass filter 226 to pass fluorescence light emitted from the reaction region 210 within a wavelength range, and circuitry 232, 238 to measure fluorescence anisotropy based on the polarization of the fluorescence light emitted relative to the excitation light. In some examples, the set of polarizers 228, 230, 234 selectively select polarization of florescence light emitted from the reaction region 210 to the first polarization and to a second polarization.

In other examples, as further illustrated by and referring to FIG. 4, a portion of the optics system is integrated on the microfluidic device 403. The portion including a bandpass filter 460 disposed on a surface of the reaction region 410 to pass fluorescence light emitted from the reaction region 410 within a wavelength range, and a set of polarizers 458, 459 disposed on the bandpass filter 460 and exposed to the first microfluidic channel 402 within the reaction region 410. The set of polarizers 458, 459 may selectively select polarization of the fluorescence light emitted from the reaction region 410 to a first polarization and to a second polarization. The portion further includes circuitry 451 coupled to the bandpass filter 460 to measure the polarization of the emitted fluorescence light relative to the first polarization and the second polarization. In some examples, the apparatus 400 may further include a light source to provide the excitation light toward the reaction region 410 (not illustrated by FIG. 4). The light source may be off-device, e.g., not on the microfluidic device 403.

As noted above and referring back to FIG. 1A, the apparatus 100 of FIG. 1A may include or be coupled to circuitry to control the flow of fluid and to eject fluid droplets of the reaction fluid 119. For example, and as illustrated by and referring to FIG. 1E, the apparatus 100 may include a controller 113 coupled to the microfluidic device 103. The controller 113 may include a processor and memory.

Memory may include a computer-readable storage medium storing a set of instructions. Computer-readable storage medium may include Read-Only Memory (ROM), Random-Access Memory (RAM), Electrically Erasable Programmable Read-Only Memory (EEPROM), flash memory, a solid state drive, physical fuses and e-fuses, and/or discrete data register sets. In some examples, computer-readable storage medium may be a non-transitory storage medium, where the term “non-transitory” does not encompass transitory propagating signals.

The processor may be a central processing unit (CPU), a semiconductor-based microprocessor, a graphics processing unit (GPU), a microcontroller, special purpose logic hardware controlled by microcode or other hardware devices suitable for retrieval and execution of instructions stored in the non-transitory computer-readable storage medium, or combinations thereof. The controller 113 may fetch, decode, and execute instructions, as described herein. As an alternative or in addition to retrieving and executing instructions, the controller may include at least one integrated circuit (IC), other control logic, other electronic circuits, or combinations thereof.

In some examples, the controller 113 may be communicatively coupled to the fluidic actuator 109 of the fluid ejector 108 to selectively actuate the fluidic actuator 109 to cause the flow of the carrier fluid coordinated with the flow of the reaction fluid to generate the fluid droplets of the reaction fluid via the cross-flow of the fluids. Alternatively and/or in addition, the controller 113 may selectively actuate the fluidic actuator 109 to eject fluid droplets of the reaction fluid. For example, in response to detecting a reaction product, the fluid droplet 121 of the reaction fluid may be ejected from the microfluidic device 103 to a substrate for further processing. As used herein, actuating the fluid ejector 108 (and other fluidic actuators or components) includes sending electrical signals, e.g., current or voltage, to fluidic actuator 109 (and other fluidic actuators or components) via electrical connects.

FIGS. 2A-2C illustrate other example apparatuses including a microfluidic device, a controller, and an optics system, in accordance with the present disclosure. The example apparatuses 200, 201, 205 each include a fluid dispensing device 233, a microfluidic device 203, a controller 213, and an optics system 216. The apparatuses 200, 201, 205 may include an implementation of and/or include similar features and components of the apparatus 100 of FIG. 1A, and are numbered accordingly. The common features and components are not repeated for ease of reference. For example, the apparatus 200 of FIG. 2A includes the microfluidic device 203 including a first microfluidic channel 202 fluidically coupled to a first reservoir 220, a second microfluidic channel 204 fluidically coupled to a second reservoir 218, and a fluid ejector 208.

As shown by FIG. 2A, an example apparatus 200 includes a fluid dispensing device 233 and the microfluidic device 203. The fluid dispensing device 233 includes a substrate transport assembly and a controller 213, and may house the microfluidic device 203. The substrate transport assembly may include a stage 237 coupled to one of the substrate 239 and the fluid dispensing device 233 to move a position of the substrate 239 with respect to the fluid dispensing device 233. The fluid dispensing device 233 may include additionally non-illustrated components, such as a mounting assembly and a power supply that provides power to the various electrical components of the fluid dispensing device 233 and the microfluidic device 203 mounted therein. The fluid dispensing device 233 may control a fluid ejector 208 of the microfluidic device 203 to dispense fluid droplets of the reaction fluid to the substrate 239. In some examples, the fluid dispensing device 233 may cause flow of the carrier fluid and the reaction fluid from the first and second reservoirs 220, 218, through the reaction region, and to the fluid ejector 208 of the microfluidic device 203, and then cause the fluid ejector 208 to eject a volume of the fluid from the microfluidic device 203 to a region of the substrate 239.

The fluid dispensing device 233 may include a jet-based fluid dispensing device used to perform chemical processes via the microfluidic device 203. Inkjet-based fluid dispensing devices may start with microliters of fluid and then dispense picoliters or nanoliters of fluid into specific regions on a substrate 239 from and using the microfluidic device 203. These regions may be specific target locations on the substrate surface, such as cavities, microwells, channels, or indentations into the substrate 239. As used herein, a microwell includes and/or refers to a column capable of storing a volume of fluid between a nanoliter and several milliliters of fluid. There may be tens, hundreds, or even thousands of dispense regions on the substrate 239, which may represent many tests on a number of samples, a number of tests on many samples, or a combination of the two. Further, multiple fluid ejectors (e.g., printheads) may dispense fluid on the substrate 239 at a time for a high-throughput design.

In some examples, the apparatus 200 includes the substrate 239. The substrate 239 may include a surface and/or material for depositing fluids from the microfluidic device 203 for further processing and/or assessment. The fluid ejector 208 may selectively eject the fluid droplets of the reaction fluid from the microfluidic device 203 to a plurality of regions of the substrate 239.

In some examples, the apparatus 200 includes stage 237 coupled to the substrate 239. The controller 213 may be communicatively coupled to the stage 237 to instruct the stage 237 to move the substrate 239 relative to the fluid ejector 208, such that the fluid ejector 208 is aligned with a select region of the plurality of regions of the substrate 239.

The various illustrated apparatuses may operate in different modes of operations. Referring to FIG. 2A, in an example first mode of operation, the controller 213 identifies a single cell, classifies the cell based on the detection or not of a reaction product, and then directs the stage 237 to position the substrate 239 under the fluid dispensing device 233 aligned with the nozzle of the fluid ejector 208 of the microfluidic device 203, and causes ejection of the cell into a particular region (e.g., well) of the substrate 239. The process is completed, and then the controller 213 may output a data map indicative of a number of cell(s) and/or cell type located in each region of the substrate. The data map may be output to external control circuitry, such as for further processing of the cells. In the first mode or another mode of operation, the controller 213 may control the position of the substrate 239 to eject a type or classification of cells into a region and to eject other classes of cells, debris or other waste to a waste region of the substrate 239.

In another mode of operation, the controller 213 identifies a single cell and classifies the cell based on the detection of the reaction product, and then directs the stage 237 to position the substrate 239 under the fluid dispensing device 233 and the cells are ejected into particular groups of regions (e.g., groups of wells) which are grouped by cell classification. The controller 213 may output a data map indicative of a number of cell(s) and/or particle type or classification located in each group of regions of the substrate 239. In some examples, the cell classification may be based on different reaction products formed, such as for testing for secretion of different target molecules by cells in parallel or sequentially.

FIG. 2B illustrates an example apparatus 201 including a microfluidic device 203 and an optics system 216. In some examples, the optics system 216 may include a confocal optics system. The apparatus 201 may include an implementation of and/or include similar features and components of the apparatus 100 of FIG. 1A and is numbered accordingly. For example, the apparatus 201 includes a microfluidic device 203 with a first microfluidic channel 202 fluidically coupled to a first reservoir, a second microfluidic channel fluidically coupled to a second reservoir, and a fluid ejector. For illustrative purposes, FIG. 2B is a close-up view of the reaction region 210 of the apparatus 201 and may not show all components.

The apparatus 201 includes an optics system 216 to detect a reaction product in the reaction region 210 of the first microfluidic channel 202. The optics system 216 includes a light source 236 to provide excitation light toward the reaction region 210, a set of polarizers 228, 230, 234 to polarize the excitation light from the light source 236 to a first polarization and selectively select polarization of florescence light emitted from the reaction region 210 to the first polarization and to a second polarization, a bandpass filter 226 to pass fluorescence light emitted from the reaction region 210 within a wavelength range, and circuitry 232, 238 to measure fluorescence anisotropy based on the polarization of the fluorescence light emitted (e.g., first polarization verses second polarization) relative to the excitation light. The first polarization and second polarization may be orthogonal to one another (e.g., 90 degrees different). In some examples, the first polarization is horizontal and the second polarization is vertical; however, examples are not so limited. The circuitry 232, 238 may include a first detector 238 to measure the intensity of emitted light at the first polarization and a second detector 232 to measure the intensity of emitted light at the second polarization.

The optics system 216 may be used to provide, a measure of fluorescence anisotropy (FA). For example, the FA measure may be used to detect the reaction product from the apparatus 201 (as well as the apparatus 100, 200). As the target molecules 222 are at least the same mass as the fluorescently-labeled capture reagents 212, 214, when a reaction product is formed that includes the fluorescently-labeled capture reagent 212, 214 bound to the target molecules 222, the FA is higher than when the fluorophore 212 is unbound in the reaction fluid in the reaction region 210. FA is a measurement of the changing orientation of a molecule in space, with respect to the time between the absorption and emission events. Absorption and emission indicate the spatial alignment of the dipoles of the molecule relative to the electric vector of the electromagnetic wave of excitation light and emitted light, respectively. If the fluorophore 212 is excited with a plane-polarized light (e.g., horizontally polarized light), it emits the plane polarized fluorescence with the same polarization. However, the emitted light retains some of the polarization based on how fast it is rotating in solution. The faster the orientation motion, the more depolarized the emitted light is. The slower the motion, the more the emitted light retains the polarization. For example, if between when the fluorophore absorbed the photon and when it emitted the photon, the molecule moves, the plane into which it emits the polarization may no longer match that of the excitation light.

FA may be defined as:

$$FA = \frac{{I}_{V} - {kI}_{H}}{{I}_{V} + 2 {KI}_{H}} ,$$

where I_{V} and I_{H} are light intensities of the vertical and horizontal polarization and k is a calibration constant for the detectors of the respective intensities. For an ideal system k=1. A common model for FA states that:

$$FA = \frac{{r}_{0}}{1 + τ / θ} ,$$

where r₀ is the maximum anisotropy possible (a constant), τ is the fluorescence lifetime (e.g., roughly the time between absorbing the excitation photon and emitting the emission photon), and θ is the rotational correlation time. In some examples, θ drives the change in FA. Specifically, θ=nV/RT, where R is the gas constant and T is the absolute temperature, n is the solvent viscosity (which itself scales as a negative exponential with temperature), and V is the effective molecular volume. When the fluorescently-labeled capture reagent 212, 214 binds to target molecule 222, the effective molecular volume increases, which increases the FA. That is, as the fluorophore becomes bound to target molecule 222, it becomes less mobile and less susceptible to random orientation and its FA increases. The increase in FA, overtime, may be measured and used to detect a reaction product and/or successful biochemical reaction.

The first and second polarizations are not limited to vertical and horizontal polarizations, and may be any orthogonal polarizations. The above example and various below examples may refer to vertical and horizontal polarizations for convenience.

In the particular example of FIG. 2B, the optics system 216 includes the light source 236, a polarizer 234 that polarizes excitation light from the light source 236, a set of lenses and apertures 223, 224, 235, 253 that focus the polarized light 225 from the light source 236 on the reaction region 210 where the fluorophore 212 is located, and a collection system that consists of lenses and apertures to collect light from a specific a bandpass filter 226 that blocks the excitation light and passes the expected fluorescence light, and a polarizing beam splitter 227 to split the beam into two optical paths. Each of the optical paths include polarizers 228, 230 to select the correct polarization of the light and a set of lenses 229, 231 to focus the light onto a detector 232, 238. The two detectors 232, 238 measure the first and second polarizations (e.g., vertical and horizontal polarization) relative to the excitation polarization.

As an example, the excitation light may be emitted by the light source 236, polarized by the polarizer 234 to a first polarization and passed through the lens 235 and to the dichroic beam splitter 253, which passes the polarized excitation light 225 through a pin hole 223 to an objective 224 that passes the excitation light 225 toward the reaction region 210. The polarized excitation light 225 excites fluorophores present in the reaction region 210, which emit fluorescent light. The emitted fluorescent light is passed through the pinhole 223 to collect only light from near the surface and is passed to the bandpass filter 226 that blocks the excitation light 225 and passes the expected fluorescence light that is within a wavelength range toward the polarizing beam splitter 227 to split the beam into the optical paths to the detectors 232, 238, as described above.

In some examples, the optics systems 216 may not include the polarizer 234 as the light source 236 provides a polarizing light. A variety of different light sources may be used, such as a laser and a light-emitted diode (LED), among other light sources. In other examples, the bandpass filter 226 may be replaced with a filter wheel to cycle through different wavelength ranges and for spectral multiplexing.

FIG. 2C illustrates an example apparatus 205 that includes a reagent cartridge 248. The apparatus 205 may include an implementation of and/or include similar features and components of the apparatus 200 of FIG. 2A, with the addition of the reagent cartridge 248 and additional stage 246 and is numbered accordingly. The common features and components are not repeated for ease of reference. For example, the apparatus 205 includes the fluid dispensing device 233, the microfluidic device 203 including the first and second microfluidic channels 202, 204 and the fluid ejector 208, the controller 213, and the optics system 216.

The apparatus 205 further includes reagent cartridge 248. The reagent cartridge 248 includes a microfluidic device including a plurality of reservoirs 244-1, 244-2, 244-L that each contain a respective fluorescently-labeled capture reagent, as shown by the respective fluorescently-labeled capture reagent 212, 214. Each of the plurality of reservoirs 244-1, 244-2, 244-L may be coupled to a common fluid ejector, or each to a separate dedicated fluid ejector to eject fluid including the fluorescently-labeled capture reagents from the plurality of reservoirs 244-1, 244-2, 244-L to the microfluidic device 203.

The fluorescently-labeled capture reagents may each include a different capture reagent having an affinity for a different target molecule. In some examples, each fluorescently-labeled capture reagent is labeled with the same fluorophore. In other examples, each fluorescently-labeled capture reagent is labeled with a different fluorophore. Use of the same fluorophore may allow for assessing a greater number of target molecules as compared to using different fluorophores at the same time, as there are a limited number of distinguishable fluorophores. Use of the different fluorophores may allow for detecting reaction products that bind to multiple captures reagents and/or for processing in parallel.

In some examples, a sample fluid containing the plurality of cells may be contained in the second reservoir 218 coupled to the second microfluidic channel 204. In such examples, the fluorescently-labeled capture reagents may be injected to an additional chamber located between the second reservoir 218 and the second microfluidic channel 204 such that the fluids may mix. In other examples, the sample fluid may be pre-mixed with the fluorescently-labeled capture reagents 214 on the reagent cartridge 248 or mixed in the second microfluidic channel 204. A chamber, as used herein, includes and/or refers to an enclosed and/or semi-enclosed region of the microfluidic device, which may be formed of an etched or micromachined portion and which may be used to perform chemical processing on fluids therein or to store fluids which the microfluidic device has chemically processed.

The fluid dispensing device 233 may further include the additional stage 246 which may move the reagent cartridge 248 relative to the microfluidic device 203 to inject the fluorescently-labeled capture reagents. In contrast, the stage 237 may move the microfluidic device 203 relative to the substrate 239. In some examples, the nozzles of fluid ejectors of the reagent cartridge 248 and the microfluidic device 203 may face other. For example, the fluid ejectors of the reagent cartridge 248 may jet the fluid upward (e.g., against gravity) to the microfluidic device 203.

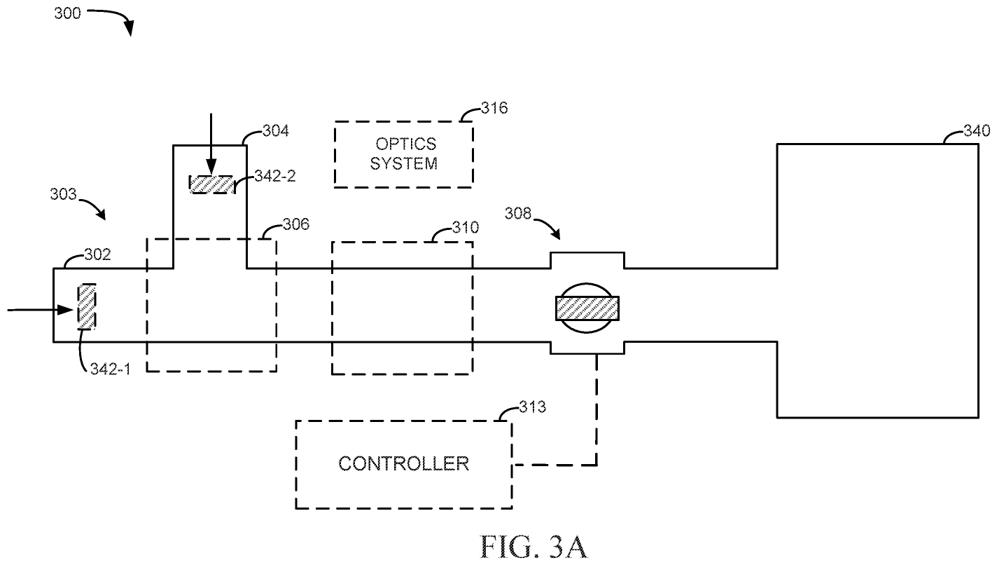

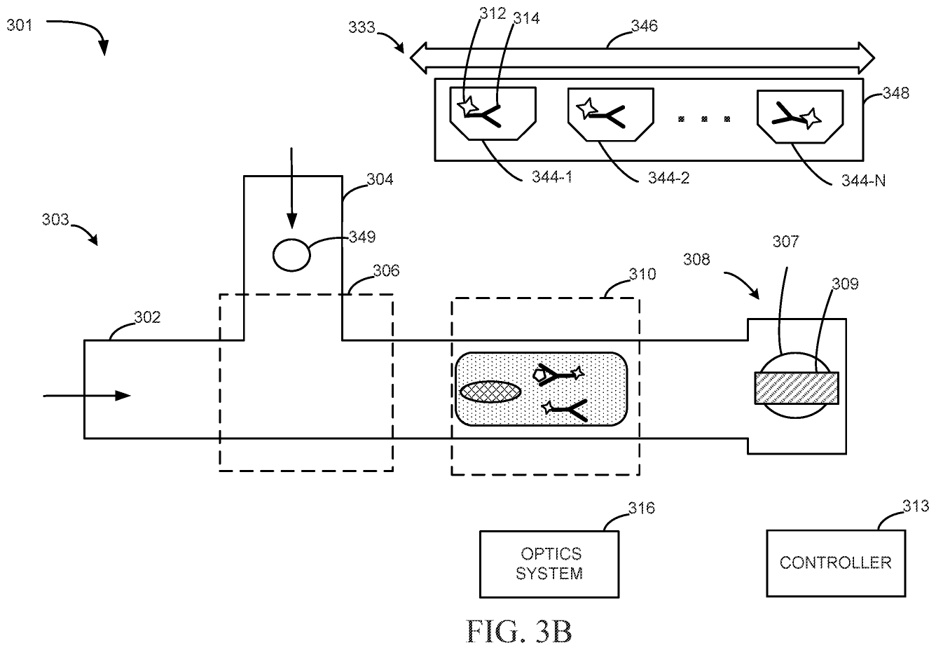

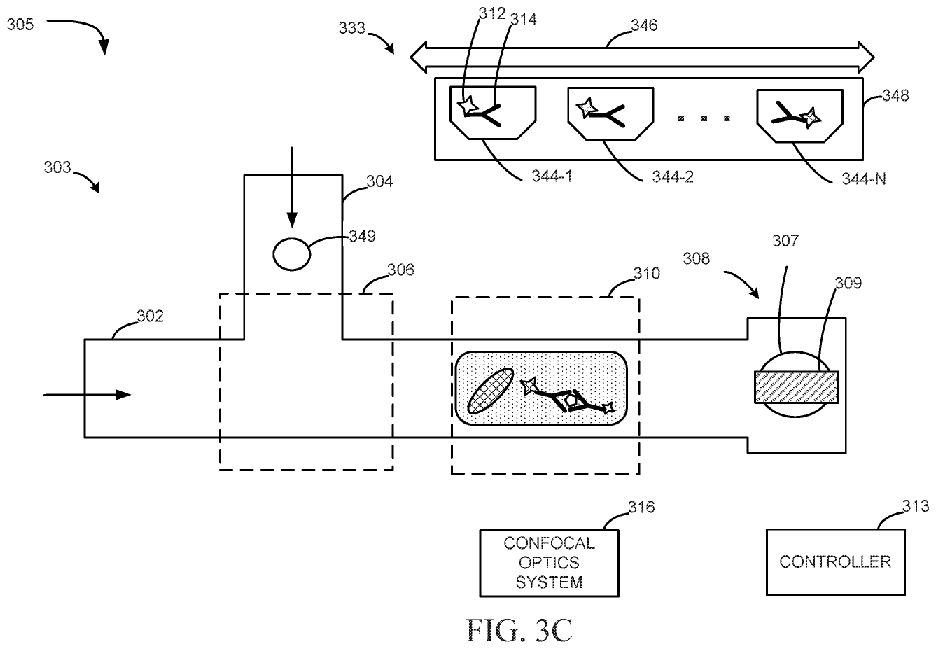

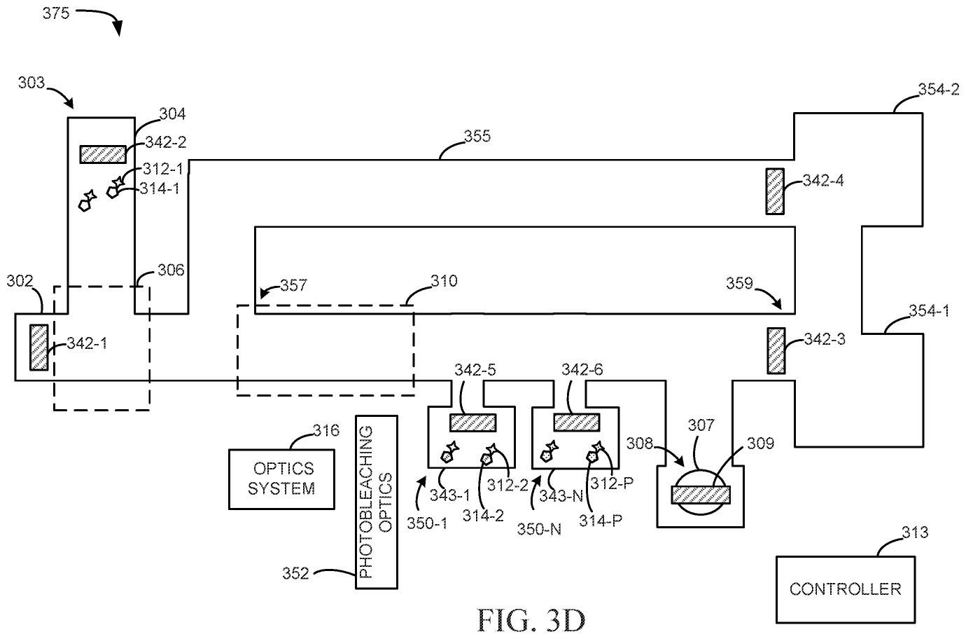

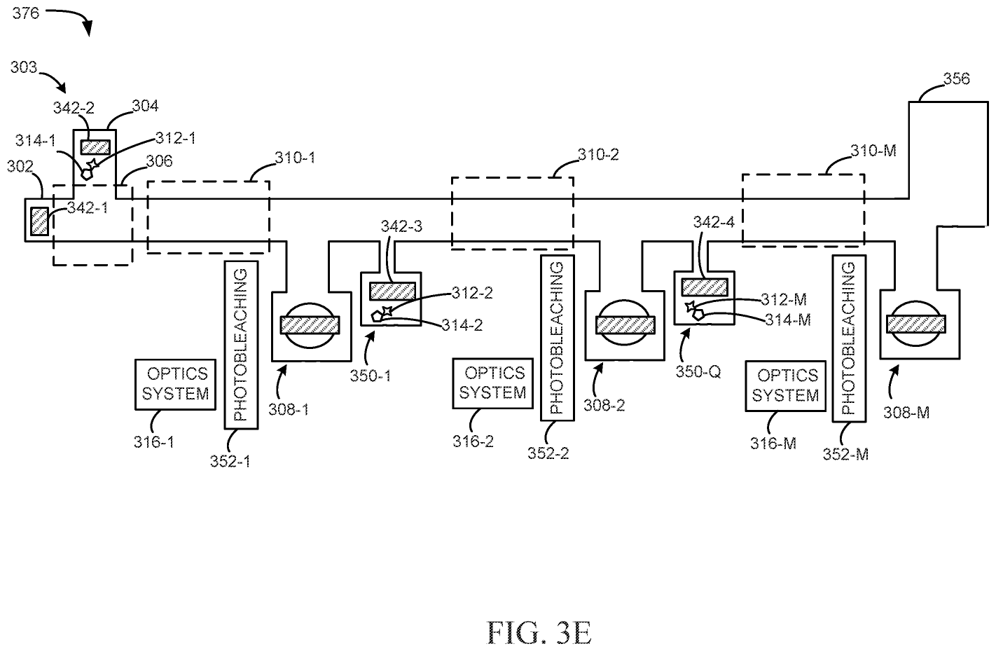

FIGS. 3A-3E illustrate different example apparatuses including a fluid droplet generator coupled to a reaction region and a fluid ejector, in accordance with the present disclosure. The apparatuses of FIGS. 3A-3E may include an implementation of and/or include similar features and components as the apparatus 100 of FIG. 1A and/or the apparatus 200 of FIG. 2A, with some variations and are numbered accordingly. For instance, each apparatus of FIGS. 3A-3E include a first microfluidic channel 302 with a reaction region 310, a second microfluidic channel 304, a fluid droplet generator 306, and a fluid ejector 308, which may form part of a microfluidic device 303. For illustrative purposes, FIGS. 3A-3E illustrate a close-up view of the apparatuses and may not show all components, such as the reservoirs.

In some examples, as illustrated by FIG. 3A, an example apparatus 300 includes a waste chamber 340. The waste chamber 340 may be fluidically coupled to the first microfluidic channel 302 downstream from the fluid ejector 308. The waste chamber 340 may include a region of the microfluidic device 303 capable of storing a volume of fluid that may not be ejected, which has been chemically processed by the apparatus 300.

The apparatus 300 may further include a controller 313 communicatively coupled to the fluid ejector 308. The controller 313 may selectively actuate the fluid ejector 308 to form the fluid droplets of the reaction fluid and flow the fluid droplets of the reaction fluid through the reaction region 310, as previously described. In operation, a fluid droplet of the reaction fluid passes by the optics system 316, which detects whether the cell(s) produce a target molecule and a reaction product is generated. If the reaction product is detected, the fluid droplet of the reaction fluid including the cell is ejected into a region of a substrate by the fluid ejector 308. If the reaction product is not detected, the fluid droplet of the reaction fluid including the cell is flown to the waste chamber 340 or to a waste region of the substrate.

The fluid droplets of the reaction fluid in the waste chamber 340 may be recycled. For example, the fluid droplets of the reaction fluid in the waste chamber 340 may be merged together and separated into products including cells and fluorescently-labeled capture reagents. In some examples, the fluid droplets of the reaction fluid in the waste chamber 340 may be centrifuged, where the cell pellet is collected and washed, and then the cells may be introduced into the second reservoir for further processing, such as with different fluorescently-labeled capture reagents. Such example apparatuses may use a single sample or reaction fluid for assessment of multiple different reaction products.

In some examples, as illustrated by FIG. 3A, the apparatus 300 may include additional fluidic actuators 342-1, 342-2. For example, the apparatus 300 may further include a first fluidic actuator 342-1 disposed within the first microfluidic channel 302 proximal to the first reservoir containing the carrier fluid, and a second fluidic actuator 342-2 disposed within the second microfluidic channel 304 proximal to the second reservoir containing the reaction fluid. Proximal, as used herein includes and/or refers to being disposed within or in line with a portion of the microfluidic device 303.

Similarly to the fluidic actuator of the fluid ejector 308, example fluidic actuators include electrodes, a fluidic pump, a magnetostrictive element, an ultrasound source, mechanical/impact driven membrane actuators, and magneto-restrictive drive actuators, among others. Example fluidic pumps include a piezo-electric pump and a resistor. A piezoelectric-based pump may include a pump assembly comprising a piezoelectric element combined with a pair of one-way valves to promote one-way directional flow through the pump and the first microfluidic channel 302 and/or second microfluidic channel 304 to which the pump is in fluid communication. In some examples, the fluidic pump of the fluidic actuators 342-1, 342-2 includes TIJ resistors.

In some examples, the controller 313 may be communicatively coupled to the fluid ejector 308, the first fluidic actuator 342-1, and the second fluidic actuator 342-2. The controller 313 may selectively actuate the fluid ejector 308, the first fluidic actuator 342-1, and the second fluidic actuator 342-2 to cause flow of the carrier fluid coordinated with flow of the reaction fluid to generate fluid droplets of the reaction fluid, selectively eject respective ones of the fluid droplets of the reaction fluid that secrete the target molecule to a substrate, and flow the remaining fluid droplets of the reaction fluid to the waste chamber 340.

For example, in operation, the fluid droplet of the reaction fluid may be formed by generating fluid flows using the first fluidic actuator 342-1 and the second fluidic actuator 342-2. In such examples, the fluidic actuator 342-1 and the second fluidic actuator 342-2 may include push and/or pull pumps which generate the cross-flows that intersect to generate the fluid droplets of the reaction fluid. The use of the additional fluidic actuators 342-1, 342-2 may be used to provide greater control of fluid droplet formation, as compared to use of the fluid ejector 308.

In some examples, as illustrated by FIGS. 3B-3C, example apparatuses 301, 305 may be used to select for multiple target molecules simultaneously or in parallel. The apparatuses 301, 305 may include an implementation of the apparatus 205 of FIG. 2C. For example, as shown by FIG. 3B, the apparatus 301 includes a microfluidic device 303 having the first and second microfluidic channels 302, 304, the fluid droplet generator 306, reaction region 310, and fluid ejector 308 with the fluidic actuator 309 and nozzle 307. The apparatus 301 further includes the fluid dispensing device 333 which houses the microfluidic device 303 and a reagent cartridge 348 with a plurality of reservoirs 344-1, 344-2, 344-N containing different fluorescently-labeled capture reagents (as shown by the respective fluorescently-labeled capture reagent 312, 314), a stage 346, an optics system 316, and a controller 313.

The controller 313 may instruct the stage 346 to move the microfluidic device 303 relative to the reagent cartridge 348 to input a select one of the different fluorescently-labeled capture reagents to the second microfluidic channel 304 via a port 349 disposed within the second microfluidic channel 304. The fluorophores may include the same fluorophore, with the different fluorescently-labeled capture reagents sequentially input and assessed. As previously described, the cells may be contained in a first reaction fluid stored in the second reservoir coupled to the second microfluidic channel 304, with the first reaction fluid and the respective fluorescently-labeled capture reagents mixing in the second microfluidic channel 304 via actuation of the fluid ejector 308 which causes fluid flow. However, examples are not so limited and the port 349 may be located on a coupled chamber and/or the second reservoir.

The apparatus 305 of FIG. 3C includes substantially the same components and features as the apparatus of FIG. 3B, with different fluorophores 312 bound to the different capture reagents 314. In such examples, as the capture reagents are bound to different fluorophores, and target molecules may be identified that bind to multiple capture reagents and/or to respective capture reagents simultaneously. In some examples, between two and six or between two and four fluorophores may be used.

In some examples, the reaction fluid may be recycled within the microfluidic device 303. For example, an apparatus 375 as shown by FIG. 3D may include a microfluidic device 303 having the first and second microfluidic channels 302, 304, the fluid droplet generator 306, the reaction region 310, and the fluid ejector 308, as previously described, with the addition of a third microfluidic channel 355 that forms a loop with the first microfluidic channel 302. The third microfluidic channel 355 may be fluidically coupled to the first microfluidic channel 302 at a first end 357 of the first microfluidic channel 302 and a second end 359 of the first microfluidic channel 302. The first end 357 may be downstream from the fluid droplet generator 306 and upstream from reaction region 310, and the second end 359 may be downstream from the fluid ejector 308.

Between the reaction region 310 and the fluid ejector 308, the microfluidic device 303 may further include a plurality of reagent injectors 350-1, 350-N. The reagent injectors 350-1, 350-N may include chambers 343-1, 343-N coupled to the first microfluidic channel 302 and fluidic actuators 342-5, 342-6 respectively disposed in the chambers 343-1, 343-N. Different fluorescently-labeled capture reagents 312-1, 312-2, 312-P, 314-1, 314-2, 314-P may be stored on the second reservoir coupled to the second microfluidic channel 304, and by the reagent injectors 350-1, 350-N. The fluidic actuators 342-5, 342-6 of the reagent injectors 350-1, 350-N may be selectively actuated by the controller 313 to control flow of fluids and to insert respective ones of the fluorescently-labeled capture reagents 312-1, 312-2, 312-P, 314-1, 314-2, 314-P. The reagent injectors 350-1, 350-N (along with fluidic actuators 342-1, 342-2, 342-3, 342-4 as further described below) may inject the respective fluorescently-labeled capture reagents 312-1, 312-2, 312-P, 314-1, 314-2, 314-P into fluid droplets of the reaction fluid already formed. As further described below, the apparatus 375 may further include photobleaching optics 352.

The microfluidic device 303 may include a plurality of fluidic actuators 342-1, 342-2, 342-3, 342-4 positioned in the first microfluidic channel 302, the second microfluidic channel 304, and the third microfluidic channel 355. Each of the fluidic actuators 342-1, 342-2, 342-3, 342-4, 342-5, 342-6 may include an implementation of the fluidic actuators 342-1, 342-2 as previously described in connection with FIG. 3A. The microfluidic device 303 may optionally include holding chambers 354-1, 354-2, which may be used to hold carrier fluid and/or reaction fluid for controlling flow.

The following describes an example operation of the apparatus 375 of FIG. 3D. The fluid droplets of the reaction fluid are generated via flow of the carrier fluid by firing the first fluidic actuator 342-1 and periodically firing the second fluidic actuator 342-2 to inject the reaction fluid as fluid droplets. For unidirectional flow of the carrier fluid, the third fluidic actuator 342-4 may be pumped with half the flow of the first fluidic actuator 342-1, thereby allowing for stagnant flow in the third microfluidic channel 355 and flow in the first microfluidic channel 302. When fluid droplets of the reaction fluid are sent back for further assessment, the third fluidic actuator 342-4 increases operational flow to exceed the first fluidic actuator 342-1 to induce flow and move the fluid droplets of the reaction fluid to the third microfluidic channel 355 and deliver the fluid droplets of the reaction fluid back to the first microfluidic channel 302 at the first end 357 of the first microfluidic channel 302.

A fluid droplet of the reaction fluid passes the optics system 316 while in the reaction region 310 to detect if the cell produces a target molecule and generates the reaction product. If the cell does not generate the reaction product, the fluorophore is photobleached when the fluid droplet of the reaction fluid passes the photobleaching optics 352. The photobleaching optics 352 may shine a laser light with a wavelength near the absorbance peak of the fluorophore. The fluid droplet of the reaction fluid is then injected with a new fluorescently-labeled capture reagent by one of the reagent injectors 350-1, 350-N. The respective reagent injectors 350-1, 350-N may be selected in a predetermined order or based on information obtained from prior assessments. The fluid droplet of the reaction fluid then travels through the loop formed by the third microfluidic channel 355 and passes through the reaction region 310 and the optics system 316 to again detect fora reaction product. If the reaction product is detected, the fluid droplet of the reaction fluid travels toward the fluid ejector 308, which fires to eject the fluid droplet of the reaction fluid out of the microfluidic device 303. If not, the fluid droplet of the reaction fluid may again be photobleached and travel through the loop. The process may be repeated until a sufficient number of cells produce reaction products, the apparatus 375 uses all the cells, and/or uses all the fluorescently-labeled capture reagents.

Such an apparatus 375 may be used to assess for multiple different target molecules at the same time, and to generate libraries of cells.

FIG. 3E illustrates an example apparatus 376 which is similar to the apparatus 375 of FIG. 3D, but in a linear arrangement. In such examples, apparatus 376 includes a first microfluidic channel 302, a second microfluidic channel 304, a fluid droplet generator 306, and a plurality of reaction regions 310-1, 310-2, 310-M and fluid ejectors 308-1, 308-2, 308-M disposed along the first microfluidic channel 302. The apparatus 376 further includes a plurality of optics systems 316-1, 316-2, 316-M and photobleaching optics 352-1, 352-2, 352-M which are disposed with each of the plurality of reaction regions 310-1, 310-2, 310-M to assess for reaction products. The apparatus 376 additionally includes a plurality of reagent injectors 350-1, 350-Q, as previously described, and an optional holding chamber 356.

In operation, a reaction fluid with cells and a first fluorescently-labeled capture reagent 312-1, 314-1 is input at the second microfluidic channel 304, with fluid droplets of the reaction fluid formed by controlling fluid flow by the first and second fluidic actuators 342-1, 342-2, as previously described. A respective fluid droplet of the reaction fluid passes through the first reaction region 310-1 and passes by the first optics system 316-1 to detect for a reaction product. If a reaction product is not detected, the first photobleaching optics 352-1 is used to photobleach the fluorophore and a second fluorescently-labeled capture reagent 312-2, 314-2 is injected to the fluid droplet of the reaction fluid via the first reagent injector 350-1. If a reaction product is detected, the fluid ejector 308-1 is fired and the fluid droplet of the reaction fluid is ejected out the nozzle by the fluid ejector 308-1. The process may repeat through (and involving fluorescently-labeled capture reagent 312-M, 314-M) the first microfluidic channel 302 until the fluid droplet of the reaction fluid is ejected or is flown to the holding chamber 356.

FIG. 4 illustrates an example microfluidic device including a portion of an optics system in a reaction region, in accordance with the present disclosure.

Similar to FIG. 1A, the microfluidic device 403 of FIG. 4 includes a first microfluidic channel 402 fluidically coupled to a first reservoir containing a carrier fluid, the first microfluidic channel 402 including a reaction region 410, and a second microfluidic channel 404 that intersects the first microfluidic channel 402 and is fluidically coupled to a second reservoir containing a reaction fluid, wherein a fluid droplet generator 406 is formed at the intersection of the first microfluidic channel 402 and the second microfluidic channel 404. A fluid ejector 408 is fluidically coupled to and disposed within the first microfluidic channel 402 and downstream from the reaction region 410 to eject fluid droplets of the reaction fluid from the first microfluidic channel 402. The fluid ejector 408 includes a nozzle 407 and a fluidic actuator 409, as previously described.

The microfluidic device 403 further includes a bandpass filter 460 disposed within the reaction region 410 and a set of polarizers 458, 459 disposed on the bandpass filter 460 and exposed to the first microfluidic channel 402 within the reaction region 410. The set of polarizers 458, 459 may be fabricated by depositing nanowires on a surface of the bandpass filter 460, the nanowires having a line width comparable to the wavelength of interest. The fabrication may include nano-lithography including deep UV, nanoimprint mask, and e-beam.

In some examples, the first microfluidic channel 402 is to pass excitation light 425 through and toward the reaction region 410 from a light source, and the set of polarizers 458, 459 are to selectively select polarization of fluorescence light emitted from the reaction region 410 as illuminated by the excitation light 425 to a first polarization (e.g., horizontal) and to a second polarization (e.g., vertical). The bandpass filter 460 may block the excitation light 425 and pass the fluorescence light emitted from the reaction region 410.

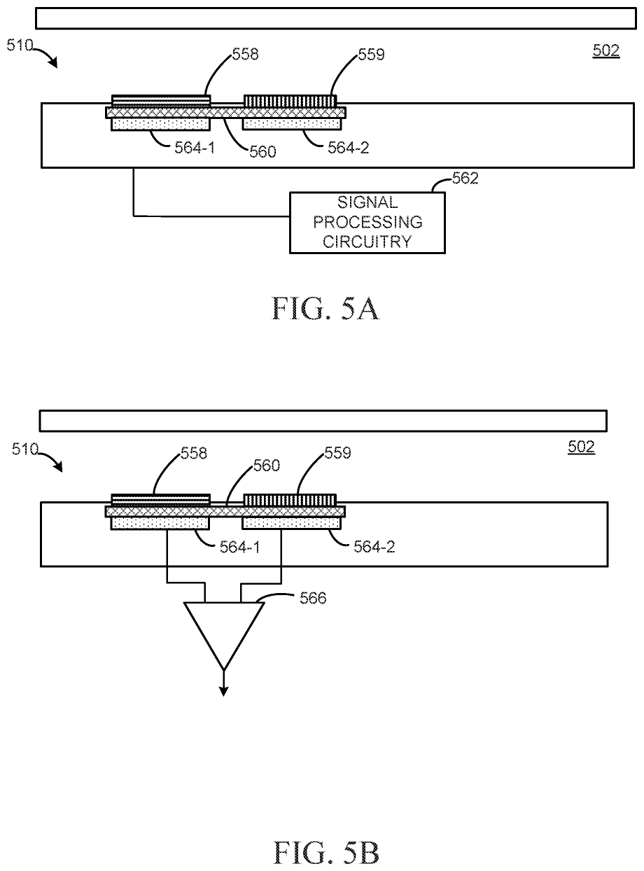

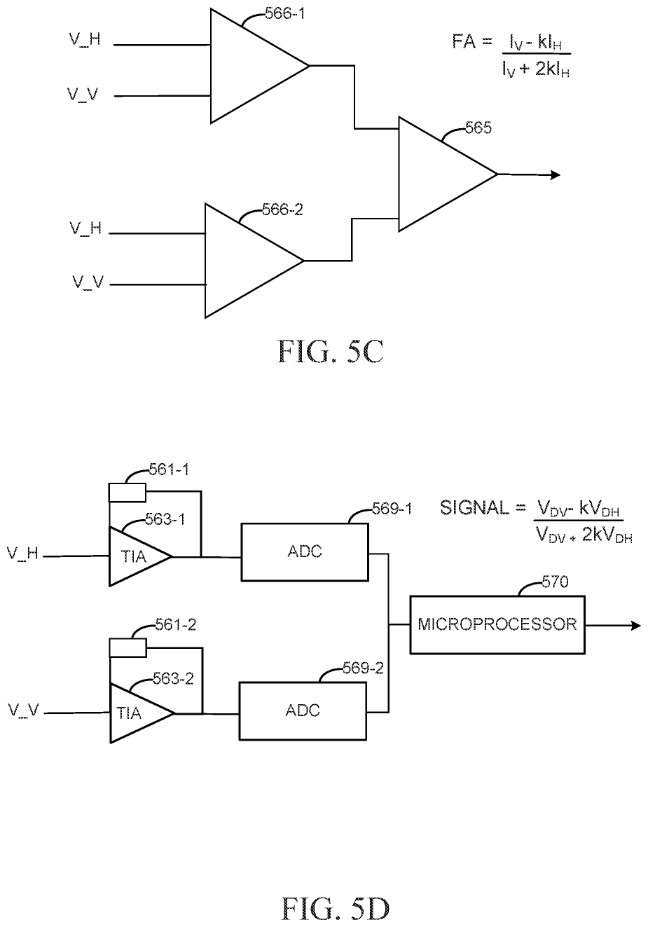

In various examples, the microfluidic device 403 may further include and/or is coupled to circuitry 451. In some examples, the microfluidic device 403 includes circuitry 451 coupled to the bandpass filter 460 to provide a FA measurement based on the polarization of the fluorescence light emitted relative to the excitation light 425. In some examples, the circuitry 451 includes a set of diodes coupled to the bandpass filter 460 and signal processing circuitry coupled to the set of diodes, as further illustrated by FIG. 5A.

As previously described, the microfluidic device 403 may form part of an apparatus 400 that further includes a controller 413. The controller 413 is communicatively coupled to the circuitry 451 and the fluid ejector 408 to cause flow of fluid, including the fluid droplets of the reaction fluid as carried by the carrier fluid, toward the reaction region 410 of the first microfluidic channel 402, and selectively eject the fluid droplets of the reaction fluid based on the FA measurement.

FIGS. 5A-5D illustrate different example microfluidic devices with immobilized capture agents and a portion of an optics system in a reaction region, in accordance with the present disclosure.

The microfluidic devices of FIGS. 5A-5D may include an implementation of and/or include similar features and components of the microfluidic device 403 of FIG. 4, with some variations for the circuitry and are numbered accordingly. For instance, each microfluidic device of FIGS. 5A-5D include a first microfluidic channel 502 with a reaction region 510, a second microfluidic channel, a fluid ejector, a bandpass filter 560, and a set of polarizers 558, 559. For illustrative purposes, FIGS. 5A-5D illustrate a close-up view of the reaction region 510 and may not illustrate the second microfluidic channel and fluid ejector.

In some examples, as illustrated by FIG. 5A, the circuitry includes a set of diodes 564-1, 564-2 coupled to the bandpass filter 560 and signal processing circuitry 562. In various examples, the microfluidic device includes multiple bandpass filters (not illustrated) which may pass light of a different wavelength range are associated with a different fluorophores. The diodes 564-1, 564-2 may include photo diodes.

The apparatus of FIG. 5B includes substantially the same components and features as the apparatus of FIG. 5A, with an example of signal processing circuitry. In some examples, the signal processing circuitry may include a differential amplifier 566 coupled to a set of diodes 564-1, 564-2 which receives current from the diodes 564-1, 564-2 and converts to a voltage signal indicative of the FA measure. In some examples, the microfluidic device includes a set of differential amplifiers, which may be for a plurality of reaction regions and/or for different fluorophores. Each differential amplifier may be coupled to a respective set of diodes and may output a signal indicative of the FA measure from the set of diodes.

FIGS. 5C-5D illustrate different example signal processing circuitry, which may be implemented in any of the microfluidic device illustrated herein, such as the microfluidic device 403 of FIG. 4. In some examples, as shown by FIG. 5C, the signal processing circuitry includes the set of differential amplifiers 566-1, 566-2, as described by FIG. 5B, which are coupled to a multiplying amplifier 565. In some examples, as shown by FIG. 5D, the signal processing circuitry include a set of differential amplifiers 561-1, 561-2, 563-1, 563-2 which convert the current from the diodes to voltage, a set of analog to digital converters (ADC) 569-1, 569-2 to convert the voltage to a digital signal, and a microprocessor 570 to provide an FA measure from the digital signals.

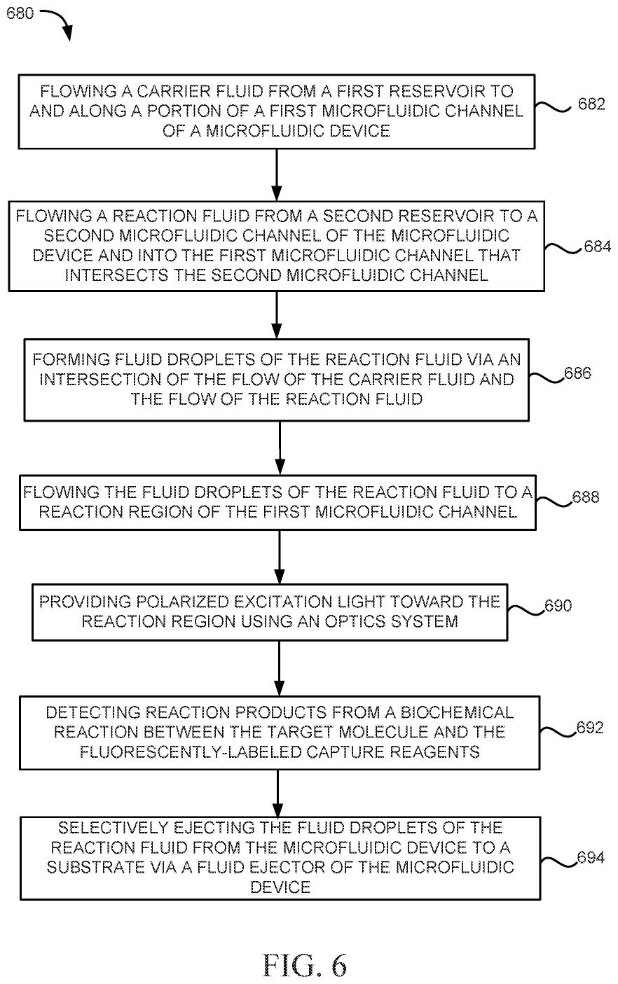

FIG. 6 illustrates an example method for detecting a reaction product, in accordance with the present disclosure.

At 682, the method 680 includes flowing a carrier fluid from a first reservoir to and along a portion of a first microfluidic channel of a microfluidic device, and, at 684, flowing a reaction fluid from a second reservoir to a second microfluidic channel of the microfluidic device and into the first microfluidic channel that intersects the second microfluidic channel, the reaction fluid including a plurality of cells and fluorescently-labeled capture reagents to form reaction products with a target molecule secreted by the plurality of cells. At 686, the method 680 further includes forming fluid droplets of the reaction fluid via an intersection of the flow of the carrier fluid and the flow of the reaction fluid, and, at 688, flowing the fluid droplets of the reaction fluid to a reaction region of the first microfluidic channel.

At 690, the method 680 includes providing polarized excitation light toward the reaction region using an optics system. At 692, the method 680 includes detecting reaction products from a biochemical reaction between the target molecule and the fluorescently-labeled capture reagents by measuring fluorescence anisotropy based on a polarization of florescence light emitted from the reaction region as illuminated by the polarized excitation light. And, at 694, the method 680 includes selectively ejecting the fluid droplets of the reaction fluid, that are associated with the detected reaction products, from the microfluidic device to a substrate via a fluid ejector of the microfluidic device.

In some examples, the method 680 may further include selectively flowing the remaining fluid droplets of the reaction fluid to one of a waste region and a recycling region. The waste region may include a waste region of a substrate or a waste chamber of the microfluidic device. The recycling region may include a holding chamber of the microfluidic device and/or a loop formed by a third microfluidic channel or additional reaction regions of the first microfluidic channel.

In various examples, the fluid droplets of the reaction fluid each include a single cell of the plurality of cells and a subset of the fluorescently-labeled capture reagents. As such, single cells may be sorted using the method 680. However, examples are not limited and may be directed to a plurality of cells within a fluid droplet of the reaction fluid.

In some examples, other methods may be directed to forming or manufacturing a microfluidic device and/or an apparatus as described herein. An example method of manufacturing may include forming a housing defining a microfluidic path including the first microfluidic channel fluidically coupled to the second microfluidic channel and a fluid ejector and/or a fluidic actuator disposed along the microfluidic path to move fluids along the microfluidic path. In some examples, the method may further include positioning circuitry for support by the housing for actuating the fluid ejector to form fluid droplets of reaction fluid and to selectively eject fluid droplets of the reaction fluid.

Any of the above described microfluidic devices may be formed of a variety of material formed in a stack. For example, a housing may formed of a plurality of different materials which are in layers, e.g., layers of substrates, in a stack. The different material layers may include a first (transparent) substrate material (e.g., top) layer and a second substrate material (e.g., bottom) layer, with etched or micromachined portioned between that form the microfluidic channels, among other components. At least one of the substrate layers may have fluidic actuators formed thereon. In some examples, the first (transparent) substrate material and the second substrate layer may have a low energy coating (e.g., a polytetrafluoroethylene (PTFE), such as Teflon™, fluorosilane, Kapton® FN, fluoroalkylsilane, 1H,1H,2H,2H-Perfluorodecyltriethoxysilane, trichloro(1H,1H,2H,2H-perfluorooctyl)silane)) proximal to and/or in contact with the microfluidic channels and the fluidic actuators, and/or a dielectric coating (e.g., a polyimide, such as Kapton®, Ethylene tetrafluoroethylene (ETFE), paralyne, alumina, silica, aluminum nitride, aluminum oxide) proximal and/or in contact with the fluidic actuators and/or the low energy coating. As used herein, a low energy coating includes and/or refers to a layer formed of a material having surface free energy less than 30 milliNewton/meter (mN/m). In some examples, the low energy coating may have a free energy of 20 mN/m, and/or may provide a contact angle hysteresis of less than about 10 degrees. The stack may additionally include a planarization layer, which may be formed of SU-8, paralyne, Polydimethylsiloxane (PDMS), acrylates, among other materials. The carrier fluid (e.g., an inert filler fluid) may be filled in the microfluidic channels. The microfluidic channels may be a height in the range of about 10 micrometers to about 2 millimeters.

In some examples, the low energy coating is formed of PTFE. In some examples, the dielectric coating may be formed of a polyimide (e.g., Kapton®) for ease of deposition. In other examples, the dielectric coating may be formed of silicon nitride. In some examples, the planarization layer may be formed of the same material as the dielectric coating, such as a polyimide, and which may reduce the number of fabrication steps. In some specific examples, the stack may include a low energy coating formed of PTFE, a dielectric coating formed of a polyimide (e.g., Kapton®), and a planarization layer formed of the polyimide (e.g., Kapton®).

Circuitry as used herein, such as the controller 213, 313, 413, includes a processor, computer readable instructions, and other electronics for communicating with and controlling the heater(s), and other components of the apparatus, such as a fluidic pump(s) and/or resistor(s), and other components. The circuitry may receive data from a host system, such as a computer, and includes memory for temporarily storing data. The data may be sent to the apparatus along an electronic, infrared, optical, or other information transfer path. A processor may be a CPU, a semiconductor-based microprocessor, a GPU, a microcontroller, special purpose logic hardware controlled by microcode or other hardware devices suitable for retrieval and/or execution of instructions stored in a memory, or combinations thereof. In addition to or alternatively to retrieving and executing instructions, the processor may include at least one IC, other control logic, other electronic circuits, or combinations thereof that include a number of electronic components for performing the function. In some examples, the circuitry includes non-transitory computer-readable storage medium that is encoded with a series of executable instructions that may be executed by the processor. Non-transitory computer-readable storage medium may be an electronic, magnetic, optical, or other physical storage device that contains or stores executable instructions. Thus, non-transitory computer-readable storage medium may be, for example, RAM, an EEPROM, a storage device, an optical disc, etc. In some examples, the computer-readable storage medium may be a non-transitory storage medium, where the term ‘non-transitory’ does not encompass transitory propagating signals.

Throughout this disclosure, use of the terms “first” and “second” does not import a temporal distinction, and is instead used to distinguish one object from another object of the same type.

A sample and/or sample fluid, as used herein, includes and/or refers to any material, collected from a subject, such as biologic material. Example samples include, but are not limited to, whole blood, blood plasma, and other body fluids, as well as tissue cell cultures obtained from humans, plants, or animals. Such samples may contain any viral or cellular material, including all prokaryotic or eukaryotic cells, viruses, bacteriophages, mycoplasmas, protoplasts, and organelles. Such biological material may comprise all types of mammalian and non-mammalian animal cells, plant cells, algae including blue-green algae, fungi, bacteria, protozoa, etc. Non-limiting examples of samples include whole blood and blood-derived products such as plasma, serum and buffy coat, urine, feces, cerebrospinal fluid or any other body fluids, tissues, cell cultures, cell suspensions, etc. Other example samples include fluids containing functionalized beads to which a portion of a biologic sample are attached. As used herein cells includes and/or refers to living cell or cells that were living and obtained from an organism, such as a basic membrane-bound unit that contains structural and functional elements.

Various terminology as used in the Specification, including the claims, connote a plain meaning in the art unless otherwise indicated. As examples, the Specification describes and/or illustrates aspects useful for implementing the claimed disclosure by way of various structure, such as circuits or circuitry selected or designed to carry out specific acts or functions, as may be recognized in the figures or the related discussion as depicted by or using terms such as blocks, device, and system, and/or other examples. It will also be appreciated that certain aspects of these blocks may also be used in combination to exemplify how operational aspects have been designed and/or arranged. Whether alone or in combination with other such blocks or circuitry including discrete circuit elements such as transistors, resistors, these above-characterized blocks may be circuits coded by fixed design and/or by configurable circuitry and/or circuit elements for carrying out such operational aspects. In certain examples, such a programmable circuit includes and/or refers includes computer circuits, including memory circuitry for storing and accessing a set of program code to be accessed/executed as instructions and/or configuration data to perform the related operation. Depending on the data-processing application, such instructions and/or data may be for implementation in logic circuitry, with the instructions as may be stored in and accessible from a memory circuit. Such instructions may be stored in and accessible from a memory via a fixed circuitry, a limited group of configuration code, or instructions characterized by way of object code.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. An apparatus comprising:
a first microfluidic channel fluidically coupled to a first reservoir containing a carrier fluid, the first microfluidic channel including a reaction region;
a fluid droplet generator including:
a portion of the first microfluidic channel; and
a second microfluidic channel that intersects the first microfluidic channel and is fluidically coupled to a second reservoir containing a reaction fluid, the reaction fluid including a plurality of cells and fluorescently-labeled capture reagents to form reaction products with a target molecule secreted by the plurality of cells; and
a fluid ejector fluidically coupled to the first microfluidic channel and disposed downstream from the reaction region of the first microfluidic channel.

2. The apparatus of claim 1, wherein the fluid ejector includes a nozzle and a fluidic actuator fluidically coupled to the nozzle, the fluidic actuator to actuate to cause flow of fluid.

3. The apparatus of claim 2, wherein the first microfluidic channel, the second microfluidic channel, and the fluid ejector are integrated on a microfluidic device, and the apparatus further includes:
a fluid dispensing device to house the microfluidic device, and including a controller communicatively coupled to the fluid ejector to selectively actuate the fluidic actuator of the fluid ejector to cause flow of the carrier fluid coordinated with flow of the reaction fluid to generate fluid droplets of the reaction fluid.

4. The apparatus of claim 3, the apparatus further including:
a substrate, wherein the fluid ejector is to selectively eject the fluid droplets of the reaction fluid from the microfluidic device to a plurality of regions of the substrate; and
a stage coupled to the substrate, wherein the controller is communicatively coupled to the stage to instruct the stage to move the substrate relative to the fluid ejector, such that the fluid ejector is aligned with a select region of the plurality of regions of the substrate.

5. The apparatus of claim 1, wherein the apparatus further includes an optics system to provide polarized excitation light toward the reaction region.

6. The apparatus of claim 5, wherein the first microfluidic channel, the second microfluidic channel, the fluid ejector, and a portion of the optics system are integrated on a microfluidic device, the portion including:
a bandpass filter disposed on a surface of reaction region to pass fluorescence light emitted from the reaction region within a wavelength range;
a set of polarizers disposed on the bandpass filter and exposed to the first microfluidic channel within the reaction region; and
circuitry coupled to the bandpass filter.

7. The apparatus of claim 5, wherein the optics system is coupled to the reaction region and includes:
a light source to provide the excitation light toward the reaction region;
a set of polarizers to polarize the excitation light from the light source to a first polarization;
a bandpass filter to pass fluorescence light emitted from the reaction region within a wavelength range; and
circuitry to measure fluorescence anisotropy based on the polarization of the fluorescence light emitted relative to the excitation light.

8. The apparatus of claim 1, the apparatus further including a waste chamber fluidically coupled to the first microfluidic channel.

9. The apparatus of claim 1, wherein:
the target molecule is a protein selected from the group consisting of: an antibody, an enzyme, a cytokine, a hormone, a metabolic product, a metabolite, a synthetic precursor, and a toxin; and
the fluorescently-labeled capture reagents is a molecule selected from the group consisting of: an antibody, an aptamer, and an antigen molecule specific to the target molecule.

10. A microfluidic device comprising:
a first microfluidic channel fluidically coupled to a first reservoir containing a carrier fluid, the first microfluidic channel including a reaction region;
a second microfluidic channel that intersects the first microfluidic channel and is fluidically coupled to a second reservoir containing a reaction fluid, the reaction fluid including a plurality of cells and fluorescently-labeled capture reagents to form reaction products with a target molecule secreted by the plurality of cells, wherein a fluid droplet generator is formed at the intersection of the first microfluidic channel and the second microfluidic channel;
a bandpass filter disposed within the reaction region;
a set of polarizers disposed on the bandpass filter and exposed to the first microfluidic channel within the reaction region; and
a fluid ejector fluidically coupled to and disposed within the first microfluidic channel and downstream from the reaction region to eject fluid droplets of the reaction fluid from the first microfluidic channel.

11. The microfluidic device of claim 10, wherein the first microfluidic channel is to pass an excitation light through and toward the reaction region from a light source, and wherein:
the set of polarizers are to selectively select polarization of fluorescence light emitted from the reaction region as illuminated by the excitation light to a first polarization and to a second polarization; and
the bandpass filter is to block the excitation light and pass the fluorescence light emitted from the reaction region.

12. The microfluidic device of claim 11, further including:
circuitry coupled to the bandpass filter to provide a fluorescence anisotropy measurement based on the polarization of the fluorescence light emitted relative to the excitation light; and
a controller communicatively coupled to the circuitry and the fluid ejector to:
cause flow of fluid, including the fluid droplets of the reaction fluid as carried by the carrier fluid, toward the reaction region of the first microfluidic channel; and
selectively eject the fluid droplets of the reaction fluid based on the fluorescence anisotropy measurement.

13. The microfluidic device of claim 12, wherein the circuitry includes a set of diodes coupled to the bandpass filter and signal processing circuitry coupled to the set of diodes.

14. A method comprising:
flowing a carrier fluid from a first reservoir to and along a portion of a first microfluidic channel of a microfluidic device;
flowing a reaction fluid from a second reservoir to a second microfluidic channel of the microfluidic device and into the first microfluidic channel that intersects the second microfluidic channel, the reaction fluid including a plurality of cells and fluorescently-labeled capture reagents to form reaction products with a target molecule secreted by the plurality of cells;
forming fluid droplets of the reaction fluid via an intersection of the flow of the carrier fluid and the flow of the reaction fluid;
flowing the fluid droplets of the reaction fluid to a reaction region of the first microfluidic channel;
providing polarized excitation light toward the reaction region using an optics system;
detecting reaction products from a biochemical reaction between the target molecule and the fluorescently-labeled capture reagents by measuring fluorescence anisotropy based on a polarization of florescence light emitted from the reaction region as illuminated by the polarized excitation light; and
selectively ejecting the fluid droplets of the reaction fluid, that are associated with the detected reaction products, from the microfluidic device to a substrate via a fluid ejector of the microfluidic device.

15. The method of claim 14, the method further including selectively flowing the remaining fluid droplets of the reaction fluid to one of a waste region and a recycling region.
